SEQUENCE LISTING (9 sequences)

SEQ ID NO: 1 - DNA, 1360 bp, Artificial Sequence
  Description of Artificial Sequence Synthetic polynucleotide
atggagacctacgtgaataagctgcacgagggaagcacctacaccgccgctgtgcagtacaatgtgctggagaaggacgatgatcctgcttccctgaccatctgggtgcccatgtttcagtctagcatgcccgccgatctgctgattaaggagctggccaacgtgaacatcctggtgaagcagatcagcaccccaaagggaccttccctgagagtgatgattaactccagaagcgccgtgctggcccagatgccctctaagttcacaatctgcgctaatgtgtccctggacgagagatccaagctggcttacgatgtgaccaccccatgcgagatcaaggcttgttctctgacctgtctgaagtccaagaatatgctgaccaccgtgaaggacctgacaatgaaaacactgaatcccacccacgatatcatcgccctgtgtgagtttgagaatatcgtgacaagcaagaaggtcatcatcccaacatacctgagatctatctctgtgaggaataaggatctgaacacactggagaatatcacaaccaccgagtttaagaacgctatcacaaacgccaagatcatcccttacagcggactgctgctggtcatcacagtgaccgataacaagggcgccttcaagtacatcaagccacagtcccagttcatcgtggatctgggcgcttacctggagaaggagagcatctactacgtgaccaccaactggaagcacacagctacaagattcgccatcaagcccatggaggaccctgatcaggctatgtctaggcgcaacccttgcaagtttgagatccggggacactgtctgaacggcaagcggtgtcacttttctcacaattactttgagtggcctcctcacgccctgctggtgcggcagaactttatgctgaatagaatcctgaagtctatggacaagtctatcgataccctgtccgagatctccggagccgctgagctggacagaaccgaggagtacgctctgggcgtggtgggcgtgctggagtcttacatcggcagcatcaacaatatcacaaagcagtccgcttgtgtggccatgtctaagctgctgacagagctgaactctgacgatatcaagaagctgcgggataacgaggagctgaattcccctaagatccgcgtgtacaacaccgtgatctcctacatcgagtccaaccgcaagaataataagcagacaatccacctgctgaagcggctgcctgccgacgtgctgaagaaaacaatcaagaacaccctggatatccacaagagcatcaccatcaataaccccaaggagtctaccgtgtccgacacaaacgatcacgccaagaacaacgacacaa

SEQ ID NO: 2 - protein, 298 aa, Human respiratory syncytial virus
MSKNKDQRTAKTLERTWDTLNHLLFISSCLYKLNLKSVAQITLSILAMIISTSLIIAAIIFIASANHKVTPTTAIIQDATSQIKNTTPTYLTQNPQLGISPSNPSEITSQITTILASTTPGVKSTLQSTTVKTKNTTTTQTQPSKPTTKQRQNKPPSKPNNDFHFEVFNFVPCSICSNNPTCWAICKRIPNKKPGKKTTTKPTKKPTLKTTKKDPKPQTTKSKEVPTTKPTEEPTINTTKTNIITTLLTSNTTGNPELTSQMETFHSTSSEGNPSPSQVSTTSEYPSQPSSPPNTPRQ

SEQ ID NO: 3 - protein, 256 aa, Human respiratory syncytial virus
METYVNKLHEGSTYTAAVQYNVLEKDDDPASLTIWVPMFQSSMPADLLIKELANVNILVKQISTPKGPSLRVMINSRSAVLAQMPSKFTICANVSLDERSKLAYDVTTPCEIKACSLTCLKSKNMLTTVKDLTMKTLNPTHDIIALCEFENIVTSKKVIIPTYLRSISVRNKDLNTLENITTTEFKNAITNAKIIPYSGLLLVITVTDNKGAFKYIKPQSQFIVDLGAYLEKESIYYVTTNWKHTATRFAIKPMED

SEQ ID NO: 4 - protein, 194 aa, Human respiratory syncytial virus
MSRRNPCKFEIRGHCLNGKRCHFSHNYFEWPPHALLVRQNFMLNRILKSMDKSIDTLSEISGAAELDRTEEYALGVVGVLESYIGSINNITKQSACVAMSKLLTELNSDDIKKLRDNEELNSPKIRVYNTVISYIESNRKNNKQTIHLLKRLPADVLKKTIKNTLDIHKSITINNPKESTVSDTNDHAKNNDTT

SEQ ID NO: 5 - protein, 391 aa, Human respiratory syncytial virus
MALSKVKLNDTLNKDQLLSSSKYTIQRSTGDSIDTPNYDVQKHINKLCGMLLITEDANHKFTGLIGMLYAMSRLGREDTIKILRDAGYHVKANGVDVTTHRQDINGKEMKFEVLTLASLTTEIQINIEIESRKSYKKMLKEMGEVAPEYRHDSPDCGMIILCIAALVITKLAAGDRSGLTAVIRRANNVLKNEMKRYKGLLPKDIANSFYEVFEKHPHFIDVFVHFGIAQSSTRGGSRVEGIFAGLFMNAYGAGQVMLRWGVLAKSVKNIMLGHASVQAEMEQVVEVYEYAQKLGGEAGFYHILNNPKASLLSLTQFPHFSSVVLGNAAGLGIMGEYRGTPRNQDLYDAAKAYAEQLKENGVINYSVLDLTAEELEAIKHQLNPKDNDVEL

SEQ ID NO: 6 - protein, 64 aa, Human respiratory syncytial virus
MENTSITIEFSSKFWPYFTLIHMITTIISLLIIISIMIAILNKLCEYNVFHNKTFELPRARVNT

SEQ ID NO: 7 - protein, 574 aa, Human respiratory syncytial virus
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 8 - protein, 298 aa, Human respiratory syncytial virus
MSKNKDQRTAKTLERTWDTLNHLLFISSCLYKLNLKSVAQITLSILAIIISTSLIIAAIIFIASANHKVTPTTAIIQDATSQIKNTTPTYLTQNPQLGISPSNPSEITSQITTILASTTPGVKSTLQSTTVKTKNTTTTQTQPSKPTTKQRQNKPPSKPNNDFHFEVFNFVPCSICSNNPTCWAICKRIPNKKPGKKTTTKPTKKPTLKTTKKDPKPQTTKSKEVPTTKPTEEPTINTTKTNIITTLLTSNTTGNPELTSQMETFHSTSSEGNPSPSQVSTTSEYPSQPSSPPNTPRQ

SEQ ID NO: 9 - DNA, 25 bp, Artificial Sequence
  Description of Artificial Sequence Synthetic oligonucleotide
gyatgntaatgarattcyttgnggg

(19) United States
(12) Patent Application Publication
(10) Pub. No.: US 2011/0091496 A1
(43) Pub. Date: Apr. 21, 2011

(54) METHODS AND COMPOSITIONS FOR THE DELIVERY OF VACCINES TO DISRUPTED EPITHELIUM

(76) Inventors: Barney S. Graham, Rockville, MD (US); Christopher B. Buck, Adelphi, MD (US); Jeffrey N. Roberts, Rockville, MD (US); Teresa R. Johnson, Gaithersburg, MD (US); John Nicewonger, Ashburn, VA (US); Rhonda Kines, Rockville, MD (US); John T. Schiller, Kensington, MD (US)

(21) Appl. No.: 12/863,572

(22) PCT Filed: Jan. 21, 2009
(86) PCT No.: PCT/US2009/031600

§ 371 Date: Oct. 13, 2010

Related U.S. Application Data
(60) Provisional application No. 61/022,324, filed on Jan. 19, 2008.

(51) Int. Cl. A61K 39/12; A61K 39/00; A61K 39/235; A61K 39/155; A61K 39/21; C07H 21/00; C12N 15/63; A61P 31/12; A61P 37/00

(52) U.S. Cl. 424/192.1; 424/184.1; 424/204.1; 424/233.1; 424/229.1; 424/211.1; 424/208.1; 536/23.1; 435/320.1

(57) ABSTRACT

The invention features immunogenic compositions and methods useful for eliciting an immune response. In preferred embodiments, papillomavirus or adenovirus vectors are used to elicit exceptionally potent antibody and T cells responses in disrupted epithelium. The methods are useful in preventing or treating a subject having a disease or an infection. In particular examples, the methods are useful for preventing or treating a viral infection.

### RELATED APPLICATIONS

This application claims the benefit of U.S. Provisional application No. 61/022,324, filed on Jan. 19, 2008. The entire contents of the aforementioned application are hereby incorporated herein by reference.

### INCORPORATION BY REFERENCE

Each of the applications and patents cited in this text, as well as each document or reference cited in each of the applications and patents (including during the prosecution of each issued patent; “application cited documents”), and each of the PCT and foreign applications or patents corresponding to and/or paragraphing priority from any of these applications and patents, and each of the documents cited or referenced in each of the application cited documents, are hereby expressly incorporated herein by reference. More generally, documents or references are cited in this text, either in a Reference List, or in the text itself; and, each of these documents or references (“herein-cited references”), as well as each document or reference cited in each of the herein-cited references (including any manufacturer's specifications, instructions, etc.), is hereby expressly incorporated herein by reference.

### STATEMENT OF RIGHTS TO INVENTIONS MADE UNDER FEDERALLY SPONSORED RESEARCH

Research supporting this application was carried out by the United States of America as represented by the Secretary, Department of Health and Human Services. This research was supported by the Intramural Research Program of the NIH, National Cancer Institute, Center for Cancer Research and the National Institute of Allergies and Infectious Disease, Vaccine Research Center. The Government has certain rights in this invention.

### BACKGROUND OF THE INVENTION

The most effective way to reduce disease and death from infectious diseases is to provide immunization to at risk or susceptible populations. Although highly effective vaccines are available against a number of pathogens, for others, infectious diseases vaccines are either not completely protective, no vaccine is available, or administration is limited.

Although immunization at one site (for example, intramuscular vaccination) can lead to effective immune responses at distant sites, immune responses are generally strongest at the site of original immunological induction. However, the female genital tract is generally considered to be a poor site for induction of both B and T cell immune responses. It has been argued that this state may have evolved to prevent infertility, which can occur due to immunological responses to sperm or to the developing embryo, both of which express various non-self antigens.

Papillomavirus-based gene delivery vectors appear to present a variety of favorable characteristics for potential use as vaccine vehicles. Systems for intracellular production of papillomaviral vectors are increasingly tractable and can be used to rapidly convert pre-existing expression plasmids into viral vector stocks with titers in excess of 10¹⁰ infections units per milliliter. A wide variety of HPV types, as well as several animal papillomavirus types to which humans are naive, have been adapted for vector production.

There remains an unmet need for vaccines that induce protective immune responses, in particular against genital infections such as HIV and herpes simplex viruses. There remains an unmet need for more effective methods of administration of immunogenic compositions.

### SUMMARY OF THE INVENTION

The present invention is based upon a novel immunization strategy. The invention is based on the finding that novel gene transfer vectors based on papillomaviruses are highly effective as genetic vaccine vehicles that are highly suitable for administration at epithelial sites. In preferred embodiments, the vectors are papillomavirus vectors, comprise of the viral L1 and L2 proteins and an encapsidated plasmid. In particular, the instant invention reports that immunogenic compositions delivered to epithelial surfaces, for example, genital vaccination, can be highly effective if the surface of the genital tract is disrupted by mechanical or chemical means.

In preferred embodiments, the invention describes papillomavirus vectors as the immunogenic compositions that are used to deliver immunogen, for example a DNA encoded antigen.

In a preferred aspect, the invention features an immunogenic composition for use in administration to a disrupted epithelial surface comprising a papillomavirus vector, wherein the papillomavirus vector comprises: L1 and L2 proteins, and a pseudogenome comprising one or more nucleic acid segments, or fragments thereof, that encode proteins or fragments thereof.

In another aspect, the invention features an immunogenic composition for use in administration to a disrupted epithelial surface comprising a papillomavirus capsid, wherein the papillomavirus capsid comprises: L1 and L2 proteins, and a vector comprising one or more nucleic acid segments, or fragments thereof, that encode proteins or fragments thereof.

In one embodiment, the L1 and L2 proteins are intracellulary assembled. In another embodiment, the one or more nucleic acid segments, or fragments thereof, encode viral proteins.

In one embodiment, the viral proteins are surface proteins.

In another embodiment, the viral proteins are internal proteins.

In another embodiment, the internal proteins are structural or regulatory proteins.

In a related embodiment, the surface proteins are viral glycoproteins.

In a further embodiment, the nucleic acid segments, or fragments thereof, encode viral proteins selected from the group consisting of: Pneumovirus, Avulavirus, Henipavirus, Morbillivirus, Respirovirus, Rubulavirus, Paramyxovirus, Metapneumovirus, Papillomavirus, Herpesvirus, Flavivirus, Poxvirus, Influenzavirus, Picornavirus, Calicivirus, Rhabdovirus, Filovirus, Bunyavirus, Orthomyxovirus, Arenavirus, Bornavirus, Reovirus, Polyomavirus, Adenovirus, Parvovirus, Hepadnavirus and Lentivirus.

In one embodiment, the Pneumovirus is Respiratory Syncytial Virus (RSV).

In another embodiment, the Papillomavirus is Human Papilloma Virus (HPV).

In a further embodiment, the Lentivirus is Human Immunodeficiency Virus (HIV).

In another embodiment, the Herpe virus is Herpes Simplex 1 or Herpes Simplex 2.

In another particular embodiment, the one or more Pneumovirus nucleic acid segments, or fragments thereof, encode one or more of a viral fusion protein (F), membrane-anchored attachment protein (Gr), matrix protein (M) or matrix protein (M2), small hydrophobic protein (SH), nucleoprotein (N), surface (HN) protein, envelope protein (E), or fragments thereof.

In a further embodiment, the one or more nucleic acid segments, or fragments thereof, comprise a fusion of the nucleic acid encoding a Pneumovirus codon-modified (M) matrix protein and codon-modified (M2) matrix protein.

In a related embodiment, the one or more nucleic acid segments, or fragments thereof, comprise a fusion of a nucleic acid encoding a codon-modified (M) matrix protein and codon-modified (M2) matrix protein of RSV. In a further embodiment, the fusion of a nucleic acid encoding a codon-modified (M) matrix protein and codon-modified (M2) matrix protein comprises SEQ ID NO: 1.

In a related embodiment, the papillomavirus is from a non-human vertebrate. In a further embodiment, the papillomavirus is selected from the group consisting of: human, ungulate, canine, lapine, avian, rodent, simian, marsupial, and marine mammal.

In another further embodiment, the papillomavirus is from a human. In still another further embodiment, the papillomavirus is selected from the group consisting of: HPV-1, HPV-2, HPV-5, HPV-6, HPV-11, HPV-18, HPV-31, HPV-45, HPV-52, and HPV-58, bovine papillomavirus-1, bovine papillomavirus-2, bovine papillomavirus-4, cottontail rabbit papillomavirs, or rhesus macaque papillomavirus.

In another embodiment of the aspect of the invention, the L1 and L2 proteins, and the vector comprising one or more nucleic acid segments, or fragments thereof, induces an immune response.

In one embodiment, the immune response is an antibody response.

In another embodiment, the immune response is a T cell immune response.

In another embodiment, the immune response is an antibody and T cell immune response.

In another embodiment, the immune response is a systemic immune response.

In another embodiment, the immune response is a mucosal immune response.

In a related embodiment, the T cell immune response comprises increased T cell cytolytic function. In another related embodiment, the T cell immune response comprises a reduction in T regulatory cells. In another embodiment, the immune response is both an antibody and a T cell immune response.

In another embodiment, the T cell immune response can modulate the pattern of the immune response.

In another further embodiment, the papillomavirus vector, comprising L1 and L2 proteins, and the pseudogenome comprising one or more genes, or fragments thereof enhances protein expression.

In another embodiment, the invention features a nucleic acid molecule encoding any one of the genes, or fragments thereof, of the aspects as described herein.

In one embodiment, the invention features an immunogenic composition comprising one or more of the nucleic acid molecules encoding the genes, or fragments thereof, of any one of the aspects as described herein, wherein the one or more nucleic acid segments, or fragments thereof, that encode viral surface proteins and a papillomavirus capsid comprising L1 and L2 proteins, enhances protein expression and modulates an immune response.

In another embodiment, the invention features a pseudogenome comprising the nucleic acid molecules encoding one or more proteins, or fragments thereof, of any one of the aspects as described herein.

In another embodiment, the invention features a plurality of vectors, each comprising the L1 and L2 proteins and nucleic acid molecule encoding one or more proteins, or fragments thereof.

In one aspect, the invention provides a method of eliciting an immune response in a subject comprising administering to the subject an immunogenic composition comprising one or more nucleic acid segments, or fragments thereof, a vector, and a pharmaceutically acceptable carrier, wherein the immunogenic composition is administered to an epithelial surface in combination with one or more agents or treatments to disrupt the epithelial surface.

In a second aspect, the invention provides a method of treating a subject having a disease or an infection comprising administering to the subject an immunogenic composition comprising one or more nucleic acid segments, or fragments thereof, a vector, and a pharmaceutically acceptable carrier, wherein the immunogenic composition is administered to an epithelial surface in combination with one or more agents or treatments to disrupt an epithelial surface.

In another aspect, the invention features a method of eliciting an immune response in a subject comprising administering to the subject an immunogenic composition comprising one or more nucleic acid segments, or fragments thereof, encapsidated in a viral vector, and a pharmaceutically acceptable carrier, wherein the immunogenic composition is administered to an epithelial surface in combination with one or more agents or treatments to disrupt the epithelial surface.

In another aspect, the invention features a method of treating a subject having a disease or an infection comprising administering to the subject an immunogenic composition comprising one or more nucleic acid segments, or fragments thereof, encapsidated in a viral vector, and a pharmaceutically acceptable carrier, wherein the immunogenic composition is administered to an epithelial surface in combination with one or more agents or treatments to disrupt an epithelial surface, thereby treating a disease or infection in a subject.

In one embodiment of any one of the above aspects, the one or more agents or treatments to disrupt an epithelial surface are administered prior to administration of the immunogenic composition.

In a further embodiment of the above-mentioned aspects, the viral vector is selected from the group consisting of: papillomavirus, poxvirus, alphavirus, adeno-associated virus, vesicular stomatitis virus, herpesvirus, rotavirus, paramyxovirus, reovirus, and enterovirus vectors. In a related embodiment, the bacterial vector is Salmonella or Bacillus Calmette-Guérin (BCG).

In another embodiment of any one of the above-mentioned aspects, the agent to disrupt the epithelial surface is a chemical agent. In a related embodiment, the chemical agent is selected from the group consisting of: a detergent, an acid and an antibody treatment. In a further embodiment, the detergent is a non-ionic or ionic detergent. In another further embodiment, the detergent is nonoxynol-9.

In another embodiment of any one of the above-mentioned aspects, the one or more treatments to disrupt the epithelial surface is a physical treatment, e.g., a cervical brush.

In another embodiment of any one of the above-mentioned aspects, the one or more treatments to disrupt the epithelial surface is a combination of a chemical treatment and a physical treatment.

In a related embodiment, the physical treatment is selected from the group consisting of: abrasion, adhesion, needle puncture, temperature treatment, electrical treatment, sonication, and radiative treatment. In a further related embodiment, the physical treatment is carried out with a device.

In another embodiment of any one of the above-mentioned aspects, the one or more nucleic acid segments, or fragments thereof, encodes viral proteins. In a related embodiment, the viral proteins are surface proteins or internal proteins. In a further related embodiment, the internal proteins are structural or regulatory proteins. In another related embodiment, the surface proteins are viral glycoproteins.

In a further embodiment, the one or more nucleic acid segments, or fragments thereof, encode viral proteins selected from the group consisting of: Pneumovirus, Avulavirus, Henipavirus, Morbillivirus, Respirovirus, Rubulavirus, Paramyxovirus, Metapneumovirus, Papillomavirus, Herpesvirus, Flavivirus, Poxvirus, Influenzavirus, Picornavirus, Calicivirus, Rhabdovirus, Filovirus, Bunyavirus, Orthomyxovirus, Arenavirus, Bornavirus, Reovirus, Polyomavirus, Adenovirus, Parvovirus, Hepadnavirus and Lentivirus.

In a particular embodiment, the Pneumovirus is Respiratory Syncytial Virus (RSV).

In another particular embodiment, the Papillomavirus is Human Papilloma Virus (HPV).

In still another embodiment, the Lentivirus is Human Immunodeficiency Virus (HIV).

In still another embodiment, the Herpesvirus is Herpes Simplex 1 or Herpes Simplex 2.

In another embodiment of the aspects of the invention as described above, the one or more nucleic acid segments, or fragments thereof, encode one or more of a viral fusion protein (F), membrane-anchored attachment protein (Gr), matrix protein (M) or matrix protein (M2), small hydrophobic protein (SH), nucleoprotein (N), surface (HN) protein, envelope protein (E), or fragments thereof.

In an embodiment of any one of the above-mentioned aspects, the one or more nucleic acid segments, or fragments thereof, comprise a fusion of the nucleic acid encoding a Pneumovirus codon-modified (M) matrix protein and codon-modified (M2) matrix protein.

In another embodiment of any one of the above-mentioned aspects, the one or more encapsidated nucleic acid segments, or fragments thereof, comprise a fusion of a nucleic acid encoding a codon-modified (M) matrix protein and codon-modified (M2) matrix protein of RSV.

In a further embodiment, the fusion of a nucleic acid encoding a codon-modified (M) matrix protein and codon-modified (M2) matrix protein comprises SEQ ID NO: 1.

In an embodiment of any one of the above-mentioned aspects, the vector is a papillomavirus vector.

In a further embodiment, the papillomavirus is from a non-human vertebrate. In a related embodiment, the papillomavirus is selected from the group consisting of: human, ungulate, canine, lapine, avian, rodent, simian, marsupial, and marine mammal. In a further related embodiment, the papillomavirus is from a human. In another particular embodiment, the papillomavirus is selected from the group consisting of: HPV-1, HPV-2, HPV-5, HPV-6, HPV-11, HPV-18, HPV-31, HPV-45, HPV-52, HPV-58, bovine papillomavirus-1, bovine papillomavirus-2, bovine papillomavirus-4, cottontail rabbit papillomavirs, and rhesus macaque papillomavirus.

In an embodiment of any one of the above-mentioned aspects, the one or more encapsidated nucleic acid segments, or fragments thereof, encode immune enhancing proteins or nucleic acids.

In a further embodiment, the immune enhancing proteins are selected from the group consisting of: cytokines, chemokines, defensins, and co-stimulatory molecules.

In an embodiment of any one of the above-mentioned aspects, the immune response is against an infection selected from a viral infection or a bacterial infection. In a further embodiment, the viral infection is selected from the group consisting of: human papillomavirus (HPV), human immunodeficiency virus (HIV), respiratory syncytial virus (RSV) and herpes simplex virus (HSV).

In an embodiment of any one of the above-mentioned aspects, the immune response is against a disease selected from the group consisting of: an infectious disease, a sexually transmitted disease, and a cancer.

In an embodiment of any one of the above-mentioned aspects, the epithelial surface is selected from the group consisting of: cervicovaginal, oral, nasal, penile, anal, epidermal and respiratory surfaces.

In an embodiment of any one of the above-mentioned aspects, the immunogenic composition is administered in a prime boost regimen. In a related embodiment, the prime boost regimen is homologous or the prime boost regimen is heterologous.

In an embodiment of any one of the above-mentioned aspects, the immunogenic composition comprising a vector transferring one or more genes or fragments thereof, and a pharmaceutically acceptable carrier, are administered together, either sequentially or in admixture. In a related embodiment, the prime boost increases the immune response.

In an embodiment of any one of the above-mentioned aspects, the subject is a mammal. In a further related embodiment, the mammal is a human.

In an embodiment of any one of the above-mentioned aspects, the immunogenic composition is administered in further combination with an adjuvant. In another embodiment, the adjuvant is selected from the group consisting of: oil emulsions, mineral compounds, bacterial products, liposomes, vertebrate gene products, nucleic acids, chemicals and immunostimulating complexes.

In still another aspect, the invention features a kit for use in a method of eliciting an immune response in a subject, the kit comprising a papillomavirus capsid, wherein the papillomavirus capsid comprises L1 and L2 proteins, and a vector comprising one or more nucleic acid segments, or fragments thereof, a pharmaceutically acceptable carrier, and instructions for use in administration to a disrupted epithelial surface.

In another aspect, the invention features a kit for use in a method of treating a subject having a disease or an infection, the kit comprising a papillomavirus vector, wherein the papillomavirus capsid comprises L1 and L2 proteins, and encapsidates one or more nucleic acid segments, or fragments thereof, a pharmaceutically acceptable carrier, and instructions for use in administration to a disrupted epithelial surface to treat a subject having a disease or an infection.

Other features and advantages of the invention will be apparent from the detailed description, and from the claims.

### BRIEF DESCRIPTION OF THE DRAWINGS

FIG. 1 is a schematic that shows the papillomavirus life cycle in epithelial tissues.

FIG. 2 is a schematic that shows the papillomavirus virion.

FIG. 3 is a schematic that shows production of papillomaviral vectors.

FIG. 4 is a schematic showing a protocol schema used to evaluate the immunogenicity of DNA delivered by papillomaviral vectors vs. gene delivery by a recombinant adenoviral vector (rAd5). The protocol schema may be used to determine whether HPV delivery of plasma DNA can induce immune responses to the expressed antigen.

FIG. 5 is a graph that shows HPV delivery of DNA induces RSV-specific tetramer+CD8+ T cells in the blood. The graphs shows Respiratory Syncytial Virus (RSV) M2 peptide-MHC tetramer-stained CD8+ T cells that were present in the blood post immunization. BALB/c H-2^{d} mice were immunized as follows: HPV16 containing the RSV M/M2 (HPV16-M/M2) plasmid followed by HVP45-containing the M/M2, given intramuscularly (IM); two doses of recombinant adenoviral vector (rAd5)ᵣAd5-M/M2 intramuscularly (IM); the plasmid HPV16-M/M2, then HPV45-M/M2, given intravaginally (IVag); two doses of rAd5-M/M2 IVag; or Mock (IM/IVag). Percent positive CD8+ T cells in whole blood is shown after primary and secondary immunization.

FIG. 6 is a graph showing antibody isotype response to vector immunization. BALB/c H-2^{d} mice were immunized with the M/M2 expressing vectors as described in FIG. 5 (HPV 16/45 IM; rAd5 IM; HPV16/45 IVag; rAd5Ivag; Mock). The results show that HPV delivery of DNA induces RSV-specific antibody in serum.

FIG. 7 is a graph showing weight loss and RSV replication in mice immunized with HPV vectors or rAd vectors. BALB/c H-2d mice were immunized with the immunization regimen as indicated in FIG. 5(HPV 16/45 IM; rAd5 IM; HPV16/45 IVag; rAdSIVag; Mock IM/IVag). Intramuscular (IM); Intravaginal (IV); Intradermal (ID). The graph on the left shows percent weight loss in the 7 days after challenge with the vectors. The panel on the right shows RSV replication at day 4 and day 7 post immunization as Log₁₀ pfu/gram in the lung after RSV challenge.

FIGS. 8 (A and B) are two graphs. (A) The graph in A shows that HPV vectors prime for early RSV-specific CD8+ T cell response post RSV challenge. The graph shows Tetramer+ CD8+ T cells present in the lung after infection with live RSV. BALB/c H-2^{d} mice were immunized with the immunization regimen indicated (HPV 16/45 IM; rAd5 IM; HPV16/45 IVag; rAdSIVag; Mock IM/IVag). Percent tetramer-positive CD8+ T cells in lung is shown on days 4 and 7 post RSV challenge. (B) is a graph showing cytotolytic T cell activity in the lung at day 7 post challenge compared to background activity on unlabeled target cells. BALB/c H-2^{d} mice were immunized with the immunization regimen indicated above (HPV 16/45 IM; rAd5 IM; HPV16/45 IVag; rAdSIVag; Mock IM/IVag).

FIG. 9 is a graph showing antibody isotype response pre and post RSV challenge. HPV 16/45 IM; rAd5 IM; HPV16/45 IVag; rAdSIVag; Mock IM/IVag vectors were used.

FIGS. 10 (A & B) is two graphs that show antibody isotype response at day 4 post RSV challenge in lung wash (A) and nasal wash (B). In both (A) and (B) BALB/c H-2^{d} mice were immunized with M/M2-expressing vectors as indicated (HPV 16/45 IM; rAd5 IM; HPV16/45 IVag; rAdSIVag; Mock IM/IVag).

FIG. 11 is a graph showing antibody isotype response in vaginal wash at day 7 post RSV challenge. HPV 16/45 IM; rAd5 IM; HPV16/45 IVag; rAdSIVag; Mock IM/IVag vectors were used.

FIG. 12 is a graph showing HPV vector priming results in Th1 response pattern in lung after RSV challenge. The mice were immunized with the regimen indicated prior to challenge (DNA-M/M2 only given IVag; HPV16-M/M2 IVag; HPV16-M/M2 administer IVag on a cotton pledget; HPV16 mock). The data indicate immunization primes for earlier production of cytokines associated with Th1-type immune responses and that cytokines associated with allergic inflammation (Th2-type responses) such as IL-4 or IL-13 are undetectable.

FIG. 13 is a schematic showing the experimental protocol used to evaluate the immunogenicity of a single dose immunization schedule comparing the HPV-16 papillomaviral vector containing a plasmid expressing the RSV M/M2 fusion protein to the plasmid given as naked DNA (DNA-M/M2). The protocol can be used to determine how HPV delivery of DNA IVag compares to naked DNA IM.

FIG. 14 is a graph showing tetramer+ CD8+ T Cells on Day 10 post immunization. BALB/c H-2^{d} mice were immunized with the immunization regimen indicated (DNA-M/M2 only given IVag; HPV16-M/M2 IVag; HPV16-M/M2 administer IVag on a cotton pledget; HPV16 mock). Percent positive CD8+ T cells is shown. Here, a single immunization induces detectable tetramer+ CD8+ T cells in the blood.

FIG. 15 is a graph showing antibody response in prechallenge serum. DNA-M/M2 IVag; HPV16-M/M2 IVag; HPV16-M/M2 cotton; and HPV16 mock vectors were used. The graph shows that HPV delivery but not naked DNA induces RSV-specific antibody prechallenge.

FIG. 16 is a graph showing tetramer+ CD8+ T Cells in the lung on Days 4, 7 & 12 post RSV challenge. The mice were immunized as follows: DNA-M/M2 only IVag; HPV16-M/M2 IVag; HPV16-M/M2 cotton; and HPV16 mock vectors were used.

FIG. 17 (A-C) are graphs. (A) is a graph showing antibody response in nasal wash at day 7 and day 12 post RSV challenge. DNA-M/M2 only IVag; HPV16-M/M2 IVag; HPV16-M/M2 cotton; and HPV16 mock vectors were used prior to RSV infection. The graph shows that HPV delivery of IVag primes for RSV-specific mucosal antibody response in nasal wash. (B) is a graph that shows antibody responses in bronchoalveolar lavage and (C) is a graph that shows antibody response in vaginal wash, both in mice primed with DNA encapsidated by HPV and challenged by RSV. These data show that priming with HPV improves the antibody response in the airway and in mucosal secretions distant from the site of infection.

FIG. 18 is a schematic showing the experimental protocol schema that was used to evaluate the need for pretreatment of the vaginal epithelium prior to intravaginal immunization. Both DNA-M/M2 delivered as naked DNA and rAd5-M/M2 were used for the single-dose immunization regimen in BALB/c H-2^{d} mice. One pretreatment that was considered in this schema was the use of Depoprovera, as shown in the schematic.

FIG. 19 is a graph showing M2 tetramer-specific blood cells on day 14 post-immunization. Mock; rAd5-M/M2; and DNA-M/M2 vectors were used. Immunization was carried out in the presence or absence of Depoprovera, as indicated. Nonoxynol-9 (N9) was used to disrupt epithelium.

FIG. 20 is a graph showing two different measurements of the M2-specific T cell response in lung on Days 4 & 7 post challenge. Tetramer+ CD8+ T cells and intracellular cytokine secretion in CD8+ T cells after peptide stimulation are shown for all conditions.

FIG. 21 shows HPV localization in the genital tract.

FIG. 22 is a schematic showing a protocol to compare the potency of papillomaviral vector delivery of a DNA-M/M2 plasmid to a dose range of the DNA-M/M2 plasmid delivered as naked DNA. In addition, M/M2 protein was included as a control for the possible contamination of M/M2 protein in the HPV preparation. The protocol can be used to determine how HPV encapsidation influences dose effect of DNA plasmid on immunogenicity.

FIGS. 23 (A and B) are two graphs. (A)shows the M2-specific T cell response by tetramer staining in lung CD8+ T cells after RSV challenge. (B) shows the antibody response at day 7. In both cases, 5 ng of DNA delivered by HPV primes better than a 10 000 fold higher dose of naked DNA.

FIG. 24 is a graph that shows luciferase expression measured by light emission in mice inoculated with DNA plasmids IVag or IM and either encapsidated by HPV or not. The data indicate that protein expression is significantly increased compared to naked DNA for 5 days after HPV delivery IVag. IM delivery of naked DNA has a peak of expression early then a low level of persistent expression. HPV delivery IM results in lower expression initially, then an increasing cumulative expression over time.

FIG. 25 is a graph that shows antibody responses induced by a single priming immunization of naked DNA IM or DNA encapsidated by HPV IVag followed by a rAd5 booster immunization IM. These data indicate that HPV delivery of vaccine antigen primes for strong antibody responses post challenge.

FIG. 26 shows the nucleotide sequence of the codon-modified matrix fusion (M/M2) gene comprising SEQ ID NO: 1.

### DETAILED DESCRIPTION OF THE INVENTION

The invention is based on the finding that immunization based on a novel method to deliver gene transfer vectors to epithelial sites. In particular, the inventors have demonstrated that delivery of gene based vaccines to disrupted epithelial surface is highly effective means to immunize subjects. In preferred embodiments, viral vectors that comprise viral structural proteins encapsidating nucleic acids that express immunogenic polypeptides are administered at disrupted epithelial sites to elicit an immune response. In particular, the instant invention reports that genital vaccination can be highly effective if the surface of the genital tract is partially disrupted by mechanical or chemical means.

The methods are useful in eliciting an immune response capable of preventing a disease or an infection. The methods are useful in treating a subject having a disease or an infection. In particular examples, the methods are useful for treating a viral or bacterial infection.

### DEFINITIONS

In this disclosure, “comprises,” “comprising,” “containing” and “having” and the like can have the meaning ascribed to them in U.S. Patent law and can mean “includes,” “including,” and the like; “consisting essentially of or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

The term “adjuvant” as used herein “refers to a compound or mixture that enhances the immune response and/or promotes the proper rate of absorption following inoculation, and, as used herein, encompasses any uptake-facilitating agent. Acceptable adjuvants include, but are not limited to, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, keyhole limpet hemocyanins, dinitrophenol, and others. The term refers to a compound or mixture that enhances the immune response and/or promotes the proper rate of absorption following inoculation, and, as used herein, encompasses any uptake-facilitating agent. Acceptable adjuvants include, but are not limited to, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, keyhole limpet hemocyanins, dinitrophenol, and others.

The term “capsid” is meant to refer to the protein shell of the virus. In particular embodiments, the capsid refers to the protein shell of the papillomavirus or adenovirus. A viral capsid may consist of multimers of oligomeric protein subunits. In certain embodiments, the capsid comprises the papillomavirus L1 and L2 proteins.

The term “cytokine” is meant to refer to a generic term for extracellular proteins or peptides that mediate cell-cell communication, often with the effect of altering the activation state of cells.

The term “disrupt” is meant to refer to compromise the barrier function of the epithelium. In certain examples, physical methods can be used to disrupt an epithelial surface. In other examples, chemical agents can be used to disrupt an epithelial surface, for example ionic or non-ionic detergents.

The term “encapsidate” refers to enclosure of a nucleic acid molecule within a structure comprising the virion structural proteins of a virus.

The term “epithelial surface” is meant to refer to a continuous sheet of one or more cellular layers that lines a vertebrate body compartment. An epithelial surface can be the skin. Epithelial surfaces according to certain embodiments of the invention can be cervicovaginal, oral, nasal, penile, anal, epidermal and respiratory surfaces The term “expression vector” is meant to refer to a vector, such as a plasmid or viral particle, which is capable of promoting expression of a foreign or heterologous nucleic acid incorporated therein. Typically, the nucleic acid to be expressed is “operably linked” to a promoter and/or enhancer, and is subject to transcription regulatory control by the promoter and/or enhancer.

The term “fragment” is meant to refer to a portion of a protein or nucleic acid that is substantially identical to a reference protein or nucleic acid. In some embodiments the fragment is a fragment of a gene. In some embodiments the fragment is a fragment of a viral gene. In some embodiments the fragment is a fragment of a viral surface protein. In some embodiments the portion can retain at least 50%, 75%, or 80%, or more preferably 90%, 95%, or even 99% of the biological activity of the reference protein or nucleic acid described herein. In other embodiments, the fragment comprises at least 5, 10, 11, 12, 13, 14, 15, 16, 17, 18, 19, or 20 amino acids of a reference protein or is a nucleic acid molecule encoding such a fragment.

The term “glycoprotein” is meant to refer to a protein that has one or more sugar molecules attached to it. In certain examples, the glycoproteins are viral glycoproteins. In other certain examples, the glycoproteins encode one or more of the fusion (F), membrane anchored attachment (Gr), matrix (M) or (M2), small hydrophobic (SH), nucleoprotein (N), surface (FIN) glycoproteins, envelope (E) glycoproteins, or fragments thereof.

The term “nucleic acid” or “nucleic acid segment” is meant to refer to an oligomer or polymer of ribonucleic acid or deoxyribonucleic acid, or analog thereof. This term includes oligomers consisting of naturally occurring bases, sugars, and intersugar (backbone) linkages as well as oligomers having non-naturally occurring portions which function similarly. Such modified or substituted oligonucleotides are often preferred over native forms because of properties such as, for example, enhanced stability in the presence of nucleases. In certain preferred embodiments, a nucleic acid segment includes a gene. The terms “L1 and L2” are meant to refer to papillomavirus capsid proteins. In preferred embodiments, the L1 and L2 proteins are intracellulary assembled.

The term “immunogenic composition” and variations thereof, as used herein is meant to refer to a composition that modulates a host's immune system. In certain embodiments, an immunogenic composition is an immunostimulatory composition. Immunogenic compositions include, but are not limited to, viruses, small molecules, peptides, polypeptides, proteins, fusion proteins, antibodies, inorganic molecules, and organic molecules.

The term “codon modified” is meant to refer to any change in codon sequence without changing protein sequence. In certain examples, codon modification increases expression of the vector components. In certain examples, the L1 and L2 proteins are codon modified. In other examples, the M, M2, G or SH proteins are codon modified.

The term “host” as used herein refers to an animal, preferably a mammal, and most preferably a human. In certain preferred embodiments, the term host cell refers to a cell that contains a heterologous nucleic acid, such as a vector, and supports the replication or expression of the nucleic acid. In certain examples, host cells can be prokaryotic cells such as E. coli, or eukaryotic cells such as yeast, insect, amphibian, avian or mammalian cells, including human cells. Exemplary host cells in include, but are not limited to, 293TT, 2930RF6, PERC.6, CHO, HEp-2, HeLa, BSC40, Vero, BHK-21, 293, C12 immortalized cell lines and primary mouse or human dendritic cells.

The term “immune response” refers to the process whereby inflammatory cells are recruited from the blood to lymphoid as well as non-lymphoid tissues via a multifactorial process that involves distinct adhesive and activation steps. In certain examples, an immune response can be a systemic or mucosal immune response, and a B cell response, a T-cell immune response or both. In certain examples, the T cell immune response comprises increased T cell cytolytic function or reduction in T regulatory cells. Inflammatory conditions cause the release of chemokines and other factors that, by upregulating and activating adhesion molecules on inflammatory cells, promote adhesion, morphological changes, and extravasation concurrent with chemotaxis through the tissues.

The term “in combination” in the context of the administration of other agents or therapies is meant to refer to the use of more than one therapy. In certain embodiments, an immunogenic composition and an agent or treatment to disrupt an epithelial surface are administered. The use of the term “in combination” does not restrict the order in which agents or therapies are administered to a subject with an infection. A first agent or therapy can be administered before (e.g., 1 minute, 45 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks), concurrently, or after (e.g., 1 minute, 45 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks) the administration of a second agent or therapy to a subject. Any additional agent or therapy can be administered in any order with the other additional treatments. Non-limiting examples of therapies that can be administered in combination with the immunogenic compositions of the invention include analgesic agents, anesthetic agents, antibiotics, or immunomodulatory agents or any other agent listed in the U.S. Pharmacopoeia and/or Physician's Desk Reference.

The term “papillomavirus” as used herein is meant to refer to any non-enveloped virus of the family Papillomaviridae.

The term “pharmaceutically acceptable” as used herein means being approved by a regulatory agency of the Federal or a state government, or listed in the U.S. Pharmacopia, European Pharmacopia or other generally recognized pharmacopia for use in animals, and more particularly in humans.

The term “promoter” refers to a DNA sequence that is recognized by RNA polymerase and initiates transcription.

The term “subject” is meant a mammal, such as a human patient or an animal (e.g., a rodent, bovine, equine, porcine, ovine, canine, feline, ferret, or other domestic mammal).

The term “vector” is meant to refer to the means by which a nucleic acid can be propagated and/or transferred between organisms, cells, or cellular components. Vectors include plasmids, viruses, bacteriophage, pro-viruses, phagemids, transposons, and artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that are not autonomously replicating. Most commonly, the vectors of the present invention are replication defective viral vectors such as recombinant adenovirus, but replication-competent viral vectors, mycobacterial vectors, bacterial vectors, or others including DNA plasmids or RNA could be used.

The term “vaccine DNA plasmid” as used herein is meant to refer to a nucleic acid sequence that encodes an immunogen from a pathogen targeted by a vaccine (i.e. RSV's M/M2 DNA plasmid).

The term “viral vector” as used herein is meant to refer to a VLP containing one or more DNA plasmids encoding one or more immunogens derived from one or more pathogens targeted by a vaccine (i.e. HPV16-M/M2 VLP DNA vaccine). The term “vector priming” is meant to refer to the delivery of a gene encoding a vaccine antigen by way of an expression vector. In certain embodiments, it means that the vector-based gene delivery will be a first exposure to the immunogenic composition, followed by one or more subsequent “booster” dose or doses of immunogenic composition.

### METHODS OF THE INVENTION

The present invention describes immunogenic compositions that are administered to an epithelial surface of a subject and elicit an immune response. Preferably, the immunogenic compositions are administered along with one or more agents or treatments to disrupt the epithelial surface. In certain embodiments, the immunogenic compositions of the invention are viral vectors.

An important feature of viral vectors is the ability to increase expression of genes encoding immunogenic proteins or polypeptides in host so that the immune system of a vertebrate induces an immune response against said protein. Accordingly, in preferred embodiments, viral vectors that comprise viral structural proteins that have encapsidated nucleic acids capable of directing expression of immunogenic proteins are administered at epithelial sites to elicit an immune response.

In one embodiment, the viral vector expresses a viral protein selected from the group consisting of, but not limited to, Pneumovirus proteins, Papillomavirus proteins, Lentivirus proteins, and Herpesvirus proteins. The Pneumovirus can be Respiratory Syncytial Virus (RSV). The Papillomavirus can be Human Papilloma Virus (HPV). The Lentivirus can be Human Immunodeficiency Virus (HIV). The Herpesvirus can be herpes simplex 1 or herpes simplex 2.

In exemplary embodiments, HIV-1 proteins, e.g., Envelope, Gag, and Pol, herpes simplex virus proteins, e.g., glycoprotein D and glycoprotein B would be useful for immunization of subjects.

In specific embodiments, the intravaginal administration of the compositions of the invention following chemical-disruption will induce both T cell and B cell immune responses including mucosal antibody responses that could prevent infection or delay disease progression caused by the virus, e.g., HIV or HSV.

Preferably, the one or more nucleic acid segments, or fragments thereof, encode one or more of a Pneumovirus fusion protein (F), membrane-anchored attachment protein (Gr), matrix protein (M) or matrix protein (M2), small hydrophobic protein (SH), nucleoprotein (N), surface (HN) protein, envelope protein (E), or fragments thereof.

The invention encompasses viral vectors or immunogenic compositions (e.g. vaccines) that they can be formulated into for protecting vertebrates (e.g. humans) against viral infection.

The methods of the invention are particularly useful for treating a subject having a disease or infection.

The methods comprise administering to the subject an immunogenic composition comprising a viral vector capable of directing the expression of one or more nucleic acid segments, and a pharmaceutically acceptable carrier, wherein the immunogenic composition is administered to an epithelial surface in combination with one or more agents or treatments that disrupt the epithelial surface, and thereby eliciting an immune response in a subject.

In related methods, the methods comprise administering to the subject an immunogenic composition comprising: a viral vector capable of directing the expression of one or more nucleic acid segments, and a pharmaceutically acceptable carrier, wherein the immunogenic composition is administered to an epithelial surface in combination with one or more agents or treatments to disrupt the epithelial surface, thereby eliciting an immune response in a subject.

The invention further encompasses a method of treating a subject having a disease or an infection. The method comprises administering to the subject an immunogenic composition comprising a viral vector capable of directing the expression of one or more nucleic acid segments, and a pharmaceutically acceptable carrier, wherein the immunogenic composition is administered to an epithelial surface in combination with one or more agents or treatments to disrupt an epithelial surface, and thereby treating a disease or infection in a subject.

The invention also features methods of eliciting an immune response in a subject comprising administering to the subject an immunogenic composition comprising a papillomavirus or adenovirus vectors, capable of directing the expression of one or more nucleic acid segments, or fragments thereof, and a pharmaceutically acceptable carrier, wherein the immunogenic composition is administered to an epithelial surface in combination with one or more agents or treatments to disrupt the epithelial surface, thereby eliciting an immune response in a subject.

### Viral Pathogens

Based on the results obtained for RSV (see the Example), HPV or adenoviral vectors expressing antigens from other viruses are contemplated in the instant invention. For example, as will be described below, HIV-1 proteins, e.g., Envelope, Gag, and Pol, herpes simplex virus proteins, e.g., glycoprotein D and glycoprotein B would be useful for immunization of subjects.

In certain preferred embodiments of the invention, the nucleic acid segments, or fragments thereof, encode viral proteins or fragments thereof. The nucleic acid segments, or fragments thereof, may encode viral proteins selected from the group consisting of, but not limited to, Pneumovirus, Papillomavirus, Lentivirus and Herpesvirus proteins.

In certain embodiments, the Pneumovirus protein may be Respiratory Syncytial Virus (RSV) protein M, M2, N, F, SH, HN, E, or Gr. In other embodiments, the Papillomavirus protein is Human Papilloma Virus (HPV) protein E1, E2, E4, E5, E6, or E7. In still other embodiments, the Lentivirus protein is Human Immunodeficiency Virus (HIV) protein env, pol, gag, rev, nef, or tat. In further embodiments, the Herpesvirus protein is herpes simplex virus 1 or herpes simplex 2 protein gB, gC, gD, or gE.

### Lentivirus

Lentiviruses refers to a group (or genus) of retroviruses that give rise to slowly developing disease. Viruses included within this group include HIV (human immunodeficiency virus; including HIV type 1, and HIV type 2), the etiologic agent of the human acquired immunodeficiency syndrome (AIDS); visna-maedi, which causes encephalitis (visna) or pneumonia (maedi) in sheep, the caprine arthritis-encephalitis virus, which causes immune deficiency, arthritis, and encephalopathy in goats; equine infectious anemia virus, which causes autoimmune hemolytic anemia, and encephalopathy in horses; feline immunodeficiency virus (FIV), which causes immune deficiency in cats; bovine immune deficiency virus (BIV), which causes lymphadenopathy, lymphocytosis, and possibly central nervous system infection in cattle; and simian immunodeficiency virus (SIV), which cause immune deficiency and encephalopathy in sub-human primates. Diseases caused by these viruses are characterized by a long incubation period and protracted course. Usually, the viruses latently infect monocytes and macrophages, from which they spread to other cells. HIV, FIV, and SIV also readily infect T lymphocytes (i.e., T-cells).

Lentivirus virions have bar-shaped nucleoids and contain genomes that are larger than other retroviruses. Lentiviruses use tRNA^{lys} as primer for negative-strand synthesis, rather than the tRNA^{pro} commonly used by other infectious mammalian retroviruses. The lentiviral genomes exhibit homology with each other, but not with other retroviruses (See, Davis et al., Microbiology, 4th ed., J. B. Lippincott Co., Philadelphia, Pa. [1990], pp. 1123-1151). An important factor in the disease caused by these viruses is the high mutability of the viral genome, which results in the production of mutants capable of evading the host immune response. It is also significant that they are capable of infecting non-dividing cells.

Lentiviruses depend on several viral regulatory genes in addition to the simple structural gag-pol-env genes for efficient intracellular replication. Thus, lentiviruses use more complex strategies than classical retroviruses for gene regulation and viral replication, with the packaging signals apparently spreading across the entire viral genome. These additional genes display a web of regulatory functions during the lentiviral life cycle. For example, upon HIV-1 infection, transcription is up-regulated by the expression of Tat through interaction with an RNA target (TAR) in the LTR. Expression of the full-length and spliced mRNAs is then regulated by the function of Rev which interacts with RNA elements present in the gag region and in the env region (RRE) (S. Schwartz et al., J. Virol., 66:150-159 [1992]). Nuclear export of gag-pol and env mRNAs is dependent on the Rev function. In addition to these two essential regulatory genes, a list of accessory genes, including vif, vpr, vpx, vpu, and nef, are also present in the viral genome and their effects on efficient virus production and infectivity have been demonstrated, although they are not absolutely required for virus replication (K. and F. Wong-Staal, Microbiol. Rev., 55:193-205 (1991]; R. A. Subbramanian and E. A. Cohen, J. Virol. 68:6831-6835 [1994]; and D. Trono, Cell 82:189-192 [1995]).

HIV-1 virions contain 60% protein and 2% nucleic acid. The genome consists of two molecules of linear positive-sense single stranded RNA (held together by hydrogen bonds to form a dimer). Even within a single virion, these molecules need not be identical. Hence, genetic variation can occur through recombination between the two viral RNAs of a single virion.

The gag gene encodes a polyprotein (55 kDa) (CDS 790.2292) which is cleaved by the viral protease (see pol) to yield various core and nucelocapsid proteins. The gag coding region extends from the ATG initiation codon at nucleotide 337 to nucleotide 1837 relative to the RNA cap site. The polyprotein is translated from unspliced viral RNA. The precursor Gag protein is cleaved by protease to produce p17 (the major matrix MA protein, involved in membrane anchoring, env interaction, and nuclear transport of viral core), p24 (the core capsid CA protein), p7 (the nucleocapsid NC protein, which binds RNA), and p6 (which binds Vpr). A pair of zinc finger motifs in the NC protein binds to the major packaging signal in the viral RNA.

The gag gene may contain one or more minor packaging signals.

The pol gene (CDS est. 2085.5096) codes for a large polyprotein which is a precursor to the virion proteins providing the viral enzyme functions: protease, reverse transcriptase, and integrase. The gag and pol genes overlap by 241 nucleotides, and are in different reading frames. A slippage sequence in or upstream of the gag-pol overlap region induces an occasional ribosomal frameshift at a frequency (about 5%) which ensures that Gag proteins are made in large amounts and Pol proteins in small amounts. Initially, a gag-pol fusion protein (p190) is created as a result of the ribosomal frameshift, which does not interrupt translation. The viral protease cleaves Gag from Pol, and further digests Gag and Pol to separate the various mature proteins. In the case of Pol, the cleavage products are protease (p10), reverse transcriptase (p50), Rnase H (p15) and integrase (p31). Roughly 50% of the RT remains linked to Rnase H as a single polypeptide (p66). The principal functional form of RT is actually a heterodimer of p66 and p50. All pol gene products are found within the capsid of free HIV-1 virions.

Reverse transcriptase is responsible for the synthesis of double-stranded DNA from the viral RNA. Activity of RT is localized to the N-terminus. RT in HIV has an extremely high error rate, 1/1700 nucleotides. At the 3′ end of the pol coding region is the coding region for viral endonuclease/integrase. Integrase functions to integrate the proviral DNA in the host genome.

The env gene is located at the 3′ end of the genome, and encodes the envelope protein gp160, some of which is cleaved to yield the envelope proteins gp120 and gp41. Both function in cell recognition on the outer envelope of a released virus. The C-terminus of gp120 interacts with the viral receptor CD4 of human T lymphocytes to facilitate the viral entry into the host cell. Only a 12 amino acid sequence in gp120 is necessary for binding to CD4; the rest of the protein is mutable. The gp120 polypeptide contains nine conserved intrachain disulfide bridges and, within this scaffolding, folds into five globular domains (I-V). There are five hypervariable regions (V1-V5) whose sequences vary especially widely among HIV-1 isolates.

Regulatory genes include the tat gene that encodes Tat, a trans-activating protein, the most important activator of the LTR promoter region, and the rev gene that encodes Rev, another transactivator.

Accessory genes include the nef gene that encodes Nef, and overlaps the env gene and the 3′ LTR, the vif gene that encodes Vif, the virion infectivity factor, the vpr gene that encodes Vpr, a virion protein which accelerates the replication and cytopathic effect of HIV-1 in CD4+ T-cells, and the vpu gene that encodes Vpu.

### Herpesvirus

Herpesviruses are enveloped double stranded DNA-containing viruses in an icosahedral nucleocapsid. At least seven herpesviruses are associated with infection in humans, including herpes simplex virus type-1 (HSV-1), herpes simplex virus type-2 (HSV-2), varicella zoster virus (VZV), Epstein Barr virus (EBV), cytomegalovirus (CMV), human herpesvirus-6 (HHV-6) and human herpesvirus-7 (HHV-7).

Their are four major structural components of the virus: An electron dense core harboring the dsDNA viral genome; a protein capsid surrounding the virus core, the capsid is comprised of 162 capsomeres; an amorphous layer surrounding the capsid termed the tegument; an envelope (lipid bilayer) containing spikes that probably represent viral glycoproteins.

The viral genome is 150 kbp in size and contains single stranded nicks and gaps. It consists of two components, a long and short region flanked by inverted repeats. The “a” sequence is highly conserved and consists of variable numbers of repeat elements. The long and short components can invert relative to each other yielding four linear isomers of the viral genome.

The herpesviruses are distinguished by their biological properties: a) they encode many enzymes involved in nucleic acid metabolism, b) their replication and assembly occur in the nucleus, c) the cell is killed (lysed) as an outcome of virus infection, d) they have the capacity to enter a latent state in which only a small subset of the viral gene complement is expressed.

Among the human herpesviruses, at least six human herpesviruses have been described. These include: Herpes simplex virus type 1 (HSV-1), Herpes simplex virus type two (HSV-2), Varicella zoster virus (VZV), Cytomegalovirus (CMV), Epstein-Barr virus (EBV), and Human herpesvirus six (HHV -6). HSV-1 and HSV-2 share extensive nucleic acid sequence homology (approximately 50%).

In preferred embodiments, the herpesvirus is HSV-1, HSV-2, VZV, EBV, CMV, HHV-6, HHV-7 or the non-human equine herpesvirus type-1. Preferably, the herpesvirus is HSV-1 or HSV-2.

HSV transcription and protein synthesis is highly ordered. Although the absolute levels of viral protein synthesis may vary, different genes can be grouped on the basis of their requirements for synthesis. Hence, HSV genes have been subdivided into 3 broad groups based on their time and requirements for expression (alpha, beta and gamma).

Among the alpha genes, there are five alpha genes which have been identified and described as ICPs (infected cell proteins), these include ICP0, ICP4, ICP22, ICP27 and ICP47. The alpha genes are by definition expressed in the absence of viral protein synthesis and contain the sequence GyATGnTAATGArATTCyTTGnGGG upstream of their coding regions. Their peak synthesis occurs 2-4 hours post infection, but they continue to accumulate until late in infection. All alpha genes appear to function as regulatory proteins with the possible exception of ICP47.

Another group is the beta genes, the beta genes are not expressed in the absence of alpha proteins and their expression is enhanced in the presence of drugs which block DNA synthesis. They reach peak rates of synthesis 5-7 hr post infection. The genes have been subdivided into the beta 1 and beta 2 subclasses. beta 1 genes appear early after infection, but require the presence of alpha 4 protein for their synthesis. Examples of beta 1 genes include the large component of ribonucleotide reductase and the major DNA binding protein (ICP8). beta 2 genes include viral thymidine kinase (TK) and the viral DNA polymerase. beta gene synthesis immediately precedes the onset of viral DNA synthesis and most viral genes involved in viral nucleic acid metabolism appear to be beta genes.

The gamma genes are also separated into two groups: gamma 1 genes are expressed early in infection and are only minimally affected by inhibitors of DNA synthesis (example, major capsid protein). Gamma 2 genes are expressed late in infection and are not expressed in the presence of inhibitors of viral DNA synthesis.

A genes map at the termini of the long and short components and tend to cluster together. In particular, alpha genes surround the HSV origin of replication in the short region. Each alpha gene has its own promoter-regulatory region and transcription initiation and termination sites. beta and gamma genes are scattered in both the long and short components. Interestingly, the beta genes specifying the DNA polymerase and the DNA binding protein flank the origin of replication in the long region (oriL). There is little gene overlap and few instances of gene splicing for any of the HSV gene classes.

There are also essential and nonessential genes. Large numbers of viral mutants have been generated and have led to identification of genes that are essential or nonessential for HSV growth in tissue culture. essential: gB, gD, major DNA binding protein (ICP8), alpha 27 and alpha 4. nonessential: all genes in the unique short region (except for gD), dUTPase, gC, alkaline DNAse, thymidine kinase, ribonucleotide reductase, uracil DNA glycosylase.

### Respiratory Syncytial Virus (RSV)

Respiratory infections are common infections of the upper respiratory tract (e.g., nose, ears, sinuses, and throat) and lower respiratory tract (e.g., trachea, bronchial tubes, and lungs). Symptoms of upper respiratory infection include runny or stuffy nose, irritability, restlessness, poor appetite, decreased activity level, coughing, and fever. Viral upper respiratory infections cause and/or are associated with sore throats, colds, croup, and the flu. Clinical manifestations of a lower respiratory infection include shallow coughing that produces sputum in the lungs, fever, and difficulty breathing.

Among the challenges for RSV vaccine development is the young age of onset of serious disease. Human RSV is the leading cause of hospitalization for viral respiratory tract disease in infants and young children worldwide, as well as a significant source of morbidity and mortality in immunocompromised adults and in the elderly. Natural immunity does not protect against reinfection with RSV, thus presenting another challenge in vaccine design. To date, no vaccines have been approved which are able to prevent the diseases associated with RSV infection. The legacy of vaccine enhanced disease presents another challenge to RSV vaccine development. RSV may be linked to epidemics of asthma and has been identified as an exacerbating factor in nephrotic disease, cystic fibrosis, and opportunistic infections in the immunocompromised. RSV is a major cause of bronchiolitis, pneumonia, mechanical ventilation, and respiratory failure in infants in the United States. By the age of two, almost all children have been infected with RSV, and most have been infected twice. Further, children who have been hospitalized in infancy with RSV bronchiolitis are at significantly increased risk of childhood asthma and allergy (Sigurs N et al. Am J Respir Crit. Care Med 171: 137-142. 2005) until the time they reach the age of 13 years (Stein R T et al. Lancet 354:541-545, 1999). RSV is a major cause of respiratory illness in the elderly and high-risk adults. RSV infection in the elderly population causes up to 14% of community-acquired pneumonia, especially in those with underlying cardiopulmonary disease. Bone marrow transplant patients develop lower respiratory tract disease with RSV, which carries a mortality of up to 50%.

### Cancer

A disease that may be treated by the methods and compositions of the invention is cancer. Examples of cancers include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endothelio sarcoma, lymphangiosarcoma, lymphangioendothelio sarcoma, synovioma, mesothelioma, Ewing's tumor, leiomyo sarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, and retinoblastoma). Lymphoproliferative disorders are also considered to be proliferative diseases.

Vectors

In the present invention, vectors are provided which comprise nucleic acid molecule encoding any one of the nucleic acid segments, or fragments thereof as described herein, and are capable of directing the expression of one or more genes, or fragments thereof. The plasmids and vectors can be used to express a gene in a host cell.

The invention features in certain embodiments, an immunogenic composition comprising one or more of the nucleic acid segments encoding the genes, or fragments thereof, as described wherein the one or more nucleic acid segments, or fragments thereof, that encode viral surface proteins. In certain embodiments, these vectors are delivered in a a papillomavirus or adenovirus vector in order to enhance protein expression and modulates an immune response.

Accordingly, the invention comprises nucleotides that encode proteins, cloned into an expression vector. A “vector” is a vector, such as a plasmid that is capable of promoting expression, as well as replication of a nucleic acid incorporated therein. Typically, the nucleic acid to be expressed is operably linked to a promoter and/or enhancer, and is subject to transcription regulatory control by the promoter and/or enhancer.

For example, in one embodiment, the invention features nucleic acid segments, or fragments thereof, that encode viral proteins. In preferred embodiments, the nucleic acids are part of immunogenic compositions, where the immunogenic compositions are used for administration to a disrupted epithelial surface and comprise a papillomavirus capsid, where the papillomavirus capsid comprises L1 and L2 proteins, and wherein the capsid contains a vector comprising one or more nucleic acid segments, or fragments thereof, that encode viral proteins or fragments.

For example, in another embodiment, the invention features nucleic acid segments, or fragments thereof, that encode viral proteins. In preferred embodiments, the nucleic acids are part of immunogenic compositions, where the immunogenic compositions are used for administration to a disrupted epithelial surface and comprise a adenovirus capsid, where the adenovirus vector comprises adenovirus capsid proteins, e.g., fiber, and one or more nucleic acid segments, or fragments that encode viral proteins or fragments.

In one embodiment, the nucleic acid segments, or fragments thereof, encode viral proteins selected from the group consisting of, but not limited to, Pneumovirus proteins, Papillomavirus proteins, Lentivirus proteins and Herpesvirus proteins. In other preferred embodiments, the one or more nucleic acid segments, or fragments thereof, encode one or more of a Pneumovirus fusion protein (F), membrane-anchored attachment protein (Gr), matrix protein (M) or matrix protein (M2), small hydrophobic protein (SH), nucleoprotein (N), surface (FIN) protein, envelope protein (E), or fragments thereof.

In preferred embodiments, the one or more nucleic acid segments, or fragments thereof, comprise a fusion of the nucleic acid encoding a Pneumovirus codon-modified (M) matrix protein and codon-modified (M2) matrix protein. In further embodiments, the one or more nucleic acid segments, or fragments thereof, comprise a fusion of a nucleic acid encoding a codon-modified (M) matrix protein and codon-modified (M2) matrix protein of RSV.

More particularly, the fusion of a nucleic acid encoding a codon-modified (M) matrix protein and codon-modified (M2) matrix protein comprises SEQ ID NO: 1.

The invention also utilizes nucleic acid and polypeptides which encode viral fusion protein (F), membrane-anchored attachment protein (Gr), matrix protein (M) or matrix protein (M2), small hydrophobic protein (SH), nucleoprotein (N), surface (FIN) protein, envelope protein (E), or fragments thereof. In one embodiment, a F, Gr, M, M2, SH, N, FIN, or E nucleic acid or protein is at least 85%, 90%, 95%, 96%, 97%, 98% or 99% identical to SEQ ID NOs 1-8, respectively.

The nucleotide sequence of the codon-modified matrix fusion (M/M2) gene comprising SEQ ID NO: 1 is shown below:

SEQ ID NO: 1
ATGGAGACCTACGTGAATAAGCTGCACGAGGGAAGCACCTACACCGCCG
CTGTGCAGTACAATGTGCTGGAGAAGGACGATGATCCTGCTTCCCTGAC
CATCTGGGTGCCCATGTTTCAGTCTAGCATGCCCGCCGATCTGCTGATT
AAGGAGCTGGCCAACGTGAACATCCTGGTGAAGCAGATCAGCACCCCAA
AGGGACCTTCCCTGAGAGTGATGATTAACTCCAGAAGCGCCGTGCTGGC
CCAGATGCCCTCTAAGTTCACAATCTGCGCTAATGTGTCCCTGGACGAG
AGATCCAAGCTGGCTTACGATGTGACCACCCCATGCGAGATCAAGGCTT
GTTCTCTGACCTGTCTGAAGTCCAAGAATATGCTGACCACCGTGAAGGA
CCTGACAATGAAAACACTGAATCCCACCCACGATATCATCGCCCTGTGT
GAGTTTGAGAATATCGTGACAAGCAAGAAGGTCATCATCCCAACATACC
TGAGATCTATCTCTGTGAGGAATAAGGATCTGAACACACTGGAGAATAT
CACAACCACCGAGTTTAAGAACGCTATCACAAACGCCAAGATCATCCCT
TACAGCGGACTGCTGCTGGTCATCACAGTGACCGATAACAAGGGCGCCT
TCAAGTACATCAAGCCACAGTCCCAGTTCATCGTGGATCTGGGCGCTTA
CCTGGAGAAGGAGAGCATCTACTACGTGACCACCAACTGGAAGCACACA
GCTACAAGATTCGCCATCAAGCCCATGGAGGACCCTGATCAGGCTATGT
CTAGGCGCAACCCTTGCAAGTTTGAGATCCGGGGACACTGTCTGAACGG
CAAGCGGTGTCACTTTTCTCACAATTACTTTGAGTGGCCTCCTCACGCC
CTGCTGGTGCGGCAGAACTTTATGCTGAATAGAATCCTGAAGTCTATGG
ACAAGTCTATCGATACCCTGTCCGAGATCTCCGGAGCCGCTGAGCTGGA
CAGAACCGAGGAGTACGCTCTGGGCGTGGTGGGCGTGCTGGAGTCTTAC
ATCGGCAGCATCAACAATATCACAAAGCAGTCCGCTTGTGTGGCCATGT
CTAAGCTGCTGACAGAGCTGAACTCTGACGATATCAAGAAGCTGCGGGA
TAACGAGGAGCTGAATTCCCCTAAGATCCGCGTGTACAACACCGTGATC
TCCTACATCGAGTCCAACCGCAAGAATAATAAGCAGACAATCCACCTGC
TGAAGCGGCTGCCTGCCGACGTGCTGAAGAAAACAATCAAGAACACCCT
GGATATCCACAAGAGCATCACCATCAATAACCCCAAGGAGTCTACCGTG
TCCGACACAAACGATCACGCCAAGAACAACGACACAA

The amino acid sequence of the unmodified membrane anchored attachment (G) glycoprotein is shown in SEQ ID NO: 2, below:

SEQ ID NO: 2
MSKNKDQRTAKTLERTWDTLNHLLFISSCLYKLNLKSVAQITLSILAMI
ISTSLIIAAIIFIASANHKVTPTTAIIQDATSQIKNTTPTYLTQNPQLG
ISPSNPSEITSQITTILASTTPGVKSTLQSTTVKTKNTTTTQTQPSKPT
TKQRQNKPPSKPNNDFHFEVFNFVPCSICSNNPTCWAICKRIPNKKPGK
KTTTKPTKKPTLKTTKKDPKPQTTKSKEVPTTKPTEEPTINTTKTNIIT
TLLTSNTTGNPELTSQMETFHSTSSEGNPSPSQVSTTSEYPSQPSSPP
NTPRQ

The amino acid sequence of the unmodified matrix (M) gene is shown in SEQ ID NO: 3, below:

SEQ ID NO: 3
METYVNKLHEGSTYTAAVQYNVLEKDDDPASLTIWVPMFQSSMPADLLI
KELANVNILVKQISTPKGPSLRVMINSRSAVLAQMPSKFTICANVSLDE
RSKLAYDVTTPCEIKACSLTCLKSKNMLTTVKDLTMKTLNPTHDIIALC
EFENIVTSKKVIIPTYLRSISVRNKDLNTLENITTTEFKNAITNAKIIP
YSGLLLVITVTDNKGAFKYIKPQSQFIVDLGAYLEKESIYYVTTNWKHT
ATRFAIKPMED

The amino acid sequence of the unmodified matrix (M2) gene is shown in SEQ ID

NO: 4, below:

SEQ ID NO: 4
MSRRNPCKFEIRGHCLNGKRCHFSHNYFEWPPHALLVRQNFMLNRILKS
MDKSIDTLSEISGAAELDRTEEYALGVVGVLESYIGSINNITKQSACVA
MSKLLTELNSDDIKKLRDNEELNSPKIRVYNTVISYIESNRKNNKQTIH
LLKRLPADVLKKTIKNTLDIHKSITINNPKESTVSDTNDHAKNNDTT

The amino acid sequence of the unmodified nucleoprotein (N) gene is shown in SEQ ID NO: 5, below:

SEQ ID NO: 5
MALSKVKLNDTLNKDQLLSSSKYTIQRSTGDSIDTPNYDVQKHINKLCG
MLLITEDANHKFTGLIGMLYAMSRLGREDTIKILRDAGYHVKANGVDVT
THRQDINGKEMKFEVLTLASLTTEIQINIEIESRKSYKKMLKEMGEVAP
EYRHDSPDCGMIILCIAALVITKLAAGDRSGLTAVIRRANNVLKNEMKR
YKGLLPKDIANSFYEVFEKHPHFIDVFVHFGIAQSSTRGGSRVEGIFAG
LFMNAYGAGQVMLRWGVLAKSVKNIMLGHASVQAEMEQVVEVYEYAQKL
GGEAGFYHILNNPKASLLSLTQFPHFSSVVLGNAAGLGIMGEYRGTPRN
QDLYDAAKAYAEQLKENGVINYSVLDLTAEELEAIKHQLNPKDNDVEL

The amino acid sequence of the unmodified SH envelope glycoprotein is shown in SEQ ID NO: 6, below:

SEQ ID NO: 6
MENTSITIEFSSKFWPYFTLIHMITTIISLLIIISIMIAILNKLCEYNV
FHNKTFELPRARVNT

The amino acid sequence of the unmodified fusion (F) glycoprotein is shown in SEQ ID NO: 7, below:

SEQ ID NO: 7
MELLILKANAITTILTAVTFCFASGQNITEEFYQSTCSAVSKGYLSALR
TGWYTSVITIELSNIKENKCNGTDAKVKLIKQELDKYKNAVTELQLLMQ
STPPTNNRARRELPRFMNYTLNNAKKTNVTLSKKRKRRFLGFLLGVGSA
IASGVAVSKVLHLEGEVNKIKSALLSTNKAVVSLSNGVSVLTSKVLDLK
NYIDKQLLPIVNKQSCSISNIETVIEFQQKNNRLLEITREFSVNAGVTT
PVSTYMLTNSELLSLINDMPITNDQKKLMSNNVQIVRQQSYSIMSIIKE
EVLAYVVQLPLYGVIDTPCWKLHTSPLCTTNTKEGSNICLTRTDRGWYC
DNAGSVSFFPQAETCKVQSNRVFCDTMNSLTLPSEINLCNVDIFNPKYD
CKIMTSKTDVSSSVITSLGAIVSCYGKTKCTASNKNRGIIKTFSNGCDY
VSNKGMDTVSVGNTLYYVNKQEGKSLYVKGEPIINFYDPLVFPSDEFDA
SISQVNEKINQSLAFIRKSDELLHNVNAGKSTTNIMITTIIIVIIVILL
SLIAVGLLLYCKARSTPVTLSKDQLSGINNIAFSN

The amino acid sequence of the retained membrane attachment glycoprotein(Gr) is shown in SEQ ID NO: 8, below:

SEQ ID NO: 8
MSKNKDQRTAKTLERTWDTLNHLLFISSCLYKLNLKSVAQITLSILAII
ISTSLIIAAIIFIASANHKVTPTTAIIQDATSQIKNTTPTYLTQNPQLG
ISPSNPSEITSQITTILASTTPGVKSTLQSTTVKTKNTTTTQTQPSKPT
TKQRQNKPPSKPNNDFHFEVFNFVPCSICSNNPTCWAICKRIPNKKPGK
KTTTKPTKKPTLKTTKKDPKPQTTKSKEVPTTKPTEEPTINTTKTNIIT
TLLTSNTTGNPELTSQMETFHSTSSEGNPSPSQVSTTSEYPSQPSSPPN
TPRQ

In certain embodiments, the sequences are codon modified.

Codons preferred by a particular prokaryotic (for example E. coli or yeast) or eukaryotic host can be modified so as to encode the same protein, but to differ from a wild type sequence. The process of codon modification may include any sequence, generated either manually or by computer software, where some or all of the codons of the native sequence are modified. Several methods have been published (Nakamura et. al., Nucleic Acids Research 1996, 24:214-215; WO98/34640). One example is the Syngene method, a modification of the Calcgene method (R. S. Hale and G Thompson (Protein Expression and Purification Vol. 12 pp. 185-188 (1998)).

In particular preferred embodiments of the invention, in order to generate the codon modified viral genes or fragments; a proprietary, patent-pending development called GENE OPTIMIZER from GeneArt Inc. (on the world wide web at geneart.com) is employed. GENE OPTIMIZER software implements multi-parameter optimization in one single operation and taking into account the most important parameters in parallel, the software generates a total of up to 500,000 optimized variants of the desired target sequence in an evolutionary approach, and then selects the one that best suits the needed requirements. WO2004059556A3 describes methods and devices for optimizing a nucleotide sequence for the purpose of expression of a protein, incorporated by reference in its entirety herein. WO2006015789A3 describes methods for modulating gene expression by modifying the CpG content, and is incorporated by reference in its entirety herein. Gene Optimizer has advantages of use in database cloning, removal of introns, knockout of cryptic splice sites and RNA destabilizing sequence elements, increased RNA stability, adaptation of codon usage, providing extensive mutagenesis, flexible combination of functional domains, introduction of restriction sites, epitope shuffling and consideration of immune modulatory CpG motifs. In addition, the F sequence was evaluated and modified manually based on consensus amino acid sequence derived from multiple sequences present in the GenBank database, and additional nucleotide sequence modifications were made based on published algorithms to reduce the possibility of splicing events altering the protein sequence.

The nucleic acids of the invention can be expressed in a vector or plurality of vectors. The vectors can be a plurality of vectors, each comprising one or more of the codon-modified genes, or fragments thereof of the invention as described herein. Thus, there can be 1, 2, 3, 4, or more vectors, each comprising one or more of the codon-modified genes, or fragments thereof of the invention as described herein. In certain examples, two or more vectors each comprise one or more of the codon-modified genes, or fragments thereof, a polynucleotide sequence according to the invention. Any of the vectors as described herein may be suitable for driving expression of heterologous DNA in bacterial, insect, mammalian cells, and particularly human cells.

Various vectors can be employed in the methods of the invention. For example, the vector can be a replication-competent vector. Alternatively, the vector can be a replication-defective vector. Exemplary replication-competent vectors include, but are not limited to vaccinia, vescicular stomatitis virus, measles virus and other Paramyxovirus vectors, BCG, and adenovirus. Exemplary replication deficient vectors include, but are not limited to adenovirus vectors based on multiple serotypes and chimeras, alphavirus vectors such as Semliki Forest virus, Venezuelan equine encephalitis virus, or Sinbis virus, MVA or other attenuated poxvirus vectors, adeno-associated virus (AAV), vescicular stomatitis virus vectors, herpesvirus vectors, or DNA alone. For example, vectors used can be, but are not limited to, bacterial vectors, adenoviral vectors, adeno-associated viral vectors, herpes simplex virus, Venezuelan equine encephalitis, BCG, retroviral vectors, Herpesvirus vectors, alphavirus vectors, flavivirus vectors, vescicular stomatitis virus vectors, mycobacterial vectors, poxvirus vectors, and nucleic acid based vectors. The vector can be an adenoviral vector selected from, but not limited to, rAd5, rAd26, rAd 41, rAd6, rAd35, and adenoviruses from other species such as chimpanzee, and chimeric adenovirus constructs. Adenoviral vectors are very efficient at transducing target cells in vitro and in vivo, and can be produced at high titres. In general, transgene expression in vivo from progenitor vectors tends to be transient. Following intravenous injection, 90% of the administered vector is degraded in the liver by a non-immune mediated mechanism (Worgall et al, 1997). Further, the finding that inverted repeats present with Ad vector genomes can mediate precise genetic recombination has important implications for the development of new vectors for gene therapy approaches, including vectors with large inserts or toxic genes. The production of rep78 expressing Ad vectors represents a major step forward in development of site-specific integrating vectors. These new Ad vectors overcome a number of limitations associated with viral vector systems.

The present invention contemplates the use of gene-based replication-defective immunomodulatory vectors, including vaccine vectors. The rationale for the use of such vectors in the methods of the invention includes the control of antigenic content, the avoidance of immune suppression or rare adverse events, the avoidance of maternal immunity, the induction of both CD8+ T cell and antibody responses and the control of immune response patterns. Further, the use of this approach may allow for the protection of the lower airway by parenteral immunization that may protect against illness while allowing boosting thorough subclinical upper airway infection.

Using any of the vectors described herein, the nucleic acid molecule or fragment is operably linked to a promoter. A promoter refers to a DNA sequence that is recognized by RNA polymerase and initiates transcription. The promoter is suitable for expression in a mammalian cell or a vertebrate cell. The promoter is suitable for expression in a cell, in particular a mammalian cell, but also including yeast, bacteria, insect cells.

The expression vectors can be used to transfect, infect, or transform and can express any of the viral proteins or fragments as described above, into eukaryotic cells and/or prokaryotic cells. Thus, the invention provides for host cells which comprise a vector (or vectors) that contain nucleic acids, for example one or more nucleic acid segments, or fragments thereof, encode viral proteins, or portions thereof, and permit their expression in a host cell.

The immunogenic compositions comprising viral vectors may be used in prime boost regimens according to the methods of the invention as described herein. The immunogenic compositions may preferably be used in a prime-boost strategy to induce robust and long-lasting immune response.

In preferred embodiments, the prime boost regimens comprise a viral vector prime.

### Immunogenic Compositions

The invention features immunogenic compositions that can be administered to disrupted epithelial surfaces, using methods as described herein. The invention features immunogenic compositions that comprise viral capsids containing nucleic acid segments that encode antigenic proteins or fragments.

In preferred embodiments, the invention features immunogenic compositions for use in administration to a disrupted epithelial surface comprising a papillomavirus capsid or an adenovirus capsid, wherein the capsid contains nucleic acids.

Papillomavirus particles are comprised of the products of the L1 (Major capsid protein) and L2 (Minor capsid protein) genes. It has been shown that L1 can spontaneously self-assemble into a 60 nanometer, 72-pentamer icosahedral structure that closely resembles authentic papillomavirus virions.

Many HPV L1 and L2 DNAs have been reported in the literature and are publicly available. (See, e.g., Baker, Sequence Analysis of Papillomavirus, Genomes, pp. 321-384; Long et al, U.S. Pat. No. 5,437,931, Cole et al, J. Mol. Biol., 193:599-608 (1987); Danos et al, EMBO J., 1:231-236 (1982); Cole et al J. Viol., 38(3):991-995 (1986)), all of which are hereby incorporated by reference in their entireties. The present invention should be broadly applicable to any HPV L1 sequence. It is known to one of skill in the art that HPV L1 DNAs exhibit significant homology. Therefore, a desired HPV L1 DNA can easily be obtained, e.g., by the use of a previously reported HPV L1 DNA or a fragment thereof as a hybridization probe or as a primer during polymerization chain reaction (PCR) amplification. Indeed, numerous HPV L1 DNAs have been cloned and expressed.

In certain examples, the HPV DNA in the subject invention will be selected from: HPV-16, HPV-18, HPV-31, HPV-33, HPV-35, HPV-39, HPV-45, HPV-51, HPV-52, HPV-56, HPV-6, HPV-11, HPV-30, HPV-42, HPV-43, HPV-44, HPV-54, HPV-55, and HPV-70. However, it is understood by one of skill in the art that the subject capsid proteins may be produced using any desired HPV L1 DNA.

Papillomaviruses are a diverse group of non-enveloped DNA viruses that infect a wide range of species. Papillomaviruses have similar genomic organizations, and any pair of two PVs contains at least five homologous genes. Phylogenetic studies strongly suggest that PVs normally evolve together with their mammalian and bird host species, do not change host species, do not recombine, and have maintained their basic genomic organization for a period exceeding 100 million years. The evolution of papillomaviruses is relatively slow compared to many other virus types. The slow evolution may be attributed to the papillomavirus genome, which is composed of genetically stable double-stranded DNA that is replicated with high fidelity by the host cells DNA replication machinery. It is believed that papillomaviruses generally co-evolve with a particular species of host animal over many years.

In certain embodiments, the papillomavirus or adenovirus can be from a non-human vertebrate. In other certain embodiments, the virus is selected from, but not limited to, human, ungulate, canine, lapine, avian, rodent, simian, marsupial, and marine mammal.

In certain preferred embodiments, the virus is from a human.

Over 100 different human papillomavirus (HPV) types have been identified. It has been reported that persistent infection with a subset of sexually transmitted HPVs, including, but not limited to types 16, 18, 31, 33, 35, 39, 45, 51, 52, 56, 58, 59, and 68, may lead to the development of cervical intraepithelial neoplasia (CIN), vulvar intraepithelial neoplasia (VIN), penile intraepithelial neoplasia (PIN), and/or anal intraepithelial neoplasia (AIN). These precancerous lesions can progress to invasive cancer. HPV infection has been reported to be a necessary factor in the development of nearly all cases of cervical cancer.

The immunogenic compositions may comprise viral capsid proteins derived from more than one type of virus. For example, the viral capsid protein may be from HPV, and as HPV 16 and 18 are associated with cervical carcinomas, an immunogenic composition for cervical neoplasia may comprise VLPs of HPV 16; of HPV 18; or both HPV 16 and 18.

In certain preferred embodiments, the human papillomavirus is selected from the group consisting of: HPV-1, HPV-2, HPV-5, HPV-6, HPV-11, HPV-18, HPV-31, HPV-45, HPV-52, HPV-58, bovine papillomavirus-1, bovine papillomavirus-2, bovine papillomavirus-4, cottontail rabbit papillomavirus, or rhesus macaque papillomavirus.

A variety of neoplasia are known to be associated with papillomavirus infections. For example, HPVs 3a and 10 have been associated with flat warts. A number of HPV types have been reported to be associated with epidermodysplasia verruciformis (EV) including HPVs 3a, 5, 8, 9, 10, and 12. HPVs 1, 2, 4, and 7 have been reported to be associated with cutaneous warts and HPVs 6b, 11a, 13, and 16 are associated with lesions of the mucus membranes (see, e.g., Kremsdorf et al, J. Virol., 52:1013-1018 (1984); Beaudenon et al, Nature, 321:246-249 (1986); Heilman et al, J. Virol., 36:395-407 (1980); and DeVilliers et al, J. Virol., 40:932-935 (1981)). Thus, the immunogenic formulations may comprise a mixture of capsid proteins or fragments derived from different viral capsid protein, e.g. HPV, types depending upon the desired protection.

In preferred embodiments, the compositions useful herein contain a pharmaceutically acceptable carrier, including any suitable diluent or excipient, which includes any pharmaceutical agent that does not itself induce the production of an immune response harmful to the vertebrate receiving the composition, and which may be administered without undue toxicity. As used herein, the term “pharmaceutically acceptable” means being approved by a regulatory agency of the Federal or a state government or listed in the U.S. Pharmacopia, European Pharmacopia or other generally recognized pharmacopia for use in mammals, and more particularly in humans. These compositions can be useful as a vaccine and/or antigenic compositions for inducing a protective immune response in a vertebrate.

The immunogenic compositions of the invention induce an immune response. In certain examples, an immune response can be a systemic or mucosal immune response, or a T-cell immune response. In certain examples, the T cell immune response comprises increased T cell cytolytic function or reduction in T regulatory cells. Inflammatory conditions cause the release of chemokines and other factors that, by upregulating and activating adhesion molecules on inflammatory cells, promote adhesion, morphological changes, and extravasation concurrent with chemotaxis through the tissues.

In certain embodiments, the immune response is an antibody response.

In other certain embodiments, the immune response is a systemic immune response.

In other embodiments, the immune response is a mucosal immune response.

The immune response may be a T cell immune response. In certain examples the T cell immune response can comprise increased T cell cytolytic function. In certain examples the T cell immune response comprises a reduction in T regulatory cells. In certain examples the T cell immune response can modulate the pattern of the immune response. In other embodiments, the immune response is a T cell response and an antibody response.

In certain embodiments, the immunogenic composition of the invention as described herein enhances protein expression.

Immunogenicity can be significantly improved if an adjuvant is co-administered with the immunostimulatory composition. Adjuvants enhance the immunogenicity of an antigen but are not necessarily immunogenic themselves. Adjuvants may act by retaining the antigen locally near the site of administration to produce a depot effect facilitating a slow, sustained release of antigen to cells of the immune system. Adjuvants can also attract cells of the immune system to an antigen depot and stimulate such cells to elicit immune responses.

Immunostimulatory agents or adjuvants have been used for many years to improve the host immune responses to, for example, vaccines. Chemically, the adjuvants are a highly heterogeneous group of compounds with only one thing in common: their ability to enhance the immune response—their adjuvanticity. They are highly variable in terms of how they affect the immune system and how serious their adverse effects are due to the resultant hyperactivation of the immune system. In the instant invention, adjuvants are not considered in the setting with live attenuated vaccine compositions, but for use with the immunogenic compositions as described herein.

The mode of action of adjuvants was described by Chedid (Ann. immunol. Inst. Pasteur 136D:283.1985) as: the formation of a depot of antigen at the site of inoculation, with slow release; the presentation of antigen immunocompetent cells; and the production of various and different lymphokines (interleukins and tumor necrosis factor). Preferred adjuvants to enhance effectiveness of the composition include, but are not limited to: (1) aluminum salts (alum), such as aluminum hydroxide, aluminum phosphate, aluminum sulfate, etc; (2) oil-in-water emulsion formulations (with or without other specific immunostimulating agents such as muramyl peptides (see below) or bacterial cell wall components), such as for example (a) MF59 (PCT Publ. No. WO 90/14837), containing 5% Squalene, 0.5% Tween 80, and 0.5% Span 85 (optionally containing various amounts of MTP-PE (see below), although not required) formulated into submicron particles using a microfluidizer such as Model 110y microfluidizer (Microfluidics, Newton, Mass.), (b) SAF, containing 10% Squalane, 0.4% Tween 80, 5% pluronic-blocked polymer L121, and thr-MDP (see below) either microfluidized into a submicron emulsion or vortexed to generate a larger particle size emulsion, and (c) RIBL.™ adjuvant system (RAS), (Ribi Immunochem, Hamilton, Mo.) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (DETOX.™); (3) saponin adjuvants, such as STIMULON.™ (Cambridge Bioscience, Worcester, Mass.) may be used or particles generated therefrom such as ISCOMs (immunostimulating complexes); (4) Complete Freunds Adjuvant (CFA) and Incomplete Freunds Adjuvant (IFA); (5) cytokines, such as interleukins (e.g., IL-1, IL-2, IL-4, IL-5, IL-6, IL-7, IL-12, etc.), interferons (e.g., gamma interferon), macrophage colony stimulating factor (M-CSF), tumor necrosis factor (TNF), etc; and (6) other substances that act as immunostimulating agents to enhance the effectiveness of the composition. Alum and MF59 are preferred.

Certain adjuvants have been shown, when co-administered with vaccine antigens, to further boost the effectiveness of vaccine compositions by stimulating the immune response (see e.g. Hibberd et al, Ann. Intern. Med., 110, 955 (1989)). Examples of adjuvants which have been shown to be effective include interferon alpha, Klebsiella pneumoniae glycoprotein and interleukin-2.

Chitosans are derivatives of chitin or poly-N-acetyl-D-glucosamine in which the greater proportion of the N-acetyl groups have been removed through hydrolysis. European Patent Application 460 020 discloses pharmaceutical formulations including chitosans as mucosal absorption enhancers.

The choice of any of these adjuvants reflects a compromise between a requirement for adjuvanticity and an acceptable low level of adverse reactions.

### Dosage and Administration

The immunogenic compositions are administered in a manner compatible with the dosage formulation, and in such amount as will be therapeutically affective, protective and immunogenic.

In preferred embodiments, the immunogenic compositions are administered to an epithelial surface that has been disrupted with one or more agents or treatments as described herein.

Immunogenic compositions may be prepared as injectables, as liquid solutions, suspensions or emulsions. The active immunogenic ingredients may be mixed with pharmaceutically acceptable excipients which are compatible therewith. Such excipients may include water, saline, dextrose, glycerol, ethanol, and combinations thereof. The immunogenic compositions and vaccines may further contain auxiliary substances, such as wetting or emulsifying agents, pH buffering agents, or adjuvants to enhance the effectiveness thereof. Immunogenic compositions may be administered by injection subcutaneous or intradermal injection. The immunogenic compositions formulated according to the present invention, are preferably, in certain embodiments, formulated and delivered in a manner to evoke an immune response at mucosal surfaces. Thus, the immunogenic composition may be administered to mucosal surfaces by, for example, the vaginal, nasal or oral (intragastric) routes. Alternatively, other modes of administration including suppositories and oral formulations may be desirable. For suppositories, binders and carriers may include, for example, polyalkalene glycols or triglycerides. Such suppositories may be formed from mixtures containing the active immunogenic ingredient(s) in the range of about 0.5 to about 10%, preferably about 1 to 2%. Oral formulations may include normally employed carriers such as, pharmaceutical grades of saccharine, cellulose and magnesium carbonate. These compositions can take the form of solutions, suspensions, tablets, pills, capsules, sustained release formulations or powders and contain about 1 to 95% of the active ingredients, preferably about 20 to about 75%.

Immunogenic compositions can be administered via injections. Traditional parenteral immunization regimes are known to have a number of drawbacks. For example, many individuals possess a natural fear of injections and may experience psychological discomfort as a result.

An effective local and/or topical administration regime may be desirable. In the case of some diseases, it would be advantageous to stimulate the mucosal immune system. In order to do this, the vaccine must be applied topically to a mucosal surface. Thus, in certain cases, it would be beneficial to obtain more effective stimulation of the local mucosal immune system of the respiratory tract.

Accordingly, a number of attempts have been made to develop mucosal vaccines. One drawback, however, is that inactivated vaccines are often poorly immunogenic when given mucosally. In order to overcome this problem, different approaches to improving the immunogenicity of vaccines given orally or intranasally have included the use of adjuvants (as described below), and encapsulation of the vaccine in a variety of microspheres.

The immunogenic preparations are administered in a manner compatible with the dosage formulation, and in such amount as will be therapeutically effective, immunogenic and protective. The quantity to be administered depends on the subject to be treated, including, for example, the capacity of the individual's immune system to synthesize antibodies, and, if needed, to produce a cell-mediated immune response. Precise amounts of active ingredients required to be administered depend on the judgment of the practitioner. However, suitable dosage ranges are readily determinable by one skilled in the art and may be of the order of micrograms to milligrams of the active ingredient(s) per vaccination. Suitable regimes for initial administration and booster doses are also variable, but may include an initial administration followed by subsequent booster administrations. The dosage may also depend on the route of administration and will vary according to the size of the host.

The immunogenic preparations or vaccines are administered in one or more doses as required to achieve the desired effect. Thus, the immunogenic preparations or vaccines may be administered in 1, 2, 3, 4, 5, or more doses. Further, the doses may be separated by any period of time, for example hours, days, weeks, months, and years.

The quantity to be administered depends on the subject to be treated. Precise amounts of active ingredient required to be administered depend on the judgment of the practitioner. However, suitable dosage ranges are readily determinable by one skilled in the art. Suitable regimes for initial administration and booster doses are also variable, but may include an initial administration followed by subsequent administrations. The dosage may also depend on the route of administration and will vary according to the size of the host. Prime boost regimens are contemplated in certain preferred embodiments, as described herein.

The immunogenic compositions according to the invention can be formulated as liquids or dry powders, or in the form of microspheres.

The immunogenic compositions, may be introduced into a host with a physiologically acceptable carrier and/or adjuvant. Useful carriers are well known in the art, and include, e.g., water, buffered water, 0.4% saline, 0.3% glycine, hyaluronic acid and the like. The resulting aqueous solutions may be packaged for use as is, or lyophilized, the lyophilized preparation being combined with a sterile solution prior to administration, as mentioned above. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, and the like. Acceptable adjuvants include incomplete Freund's adjuvant, aluminum phosphate, aluminum hydroxide, or alum, or any of the adjuvants mentioned herein, which are materials well known in the art.

In another embodiment, the immunogenic compositions can be delivered in an exosomal delivery system. Exosomes are small membrane vesicles that are released into the extracellular environment during fusion of multivesicular bodies with plasma membrane. Exosomes are secreted by various cell types including hematopoietic cells, normal epithelial cells and even some tumor cells. Exosomes are known to carry MHC class I, various costimulatory molecules and some tetraspanins. Recent studies have shown the potential of using native exosomes as immunologic stimulants.

Also contemplated by the invention is delivery of the immunogenic composition using nanoparticles. For example, the immunogenic compositions provided herein can contain nanoparticles having at least one or more immunogenic compositions linked thereto, e.g., linked to the surface of the nanoparticle. A composition typically includes many nanoparticles with each nanoparticle having at least one or more immunogenic compositions linked thereto. Nanoparticles can be colloidal metals. A colloidal metal includes any water-insoluble metal particle or metallic compound dispersed in liquid water. Typically, a colloid metal is a suspension of metal particles in aqueous solution. Any metal that can be made in colloidal form can be used, including gold, silver, copper, nickel, aluminum, zinc, calcium, platinum, palladium, and iron. In some cases, gold nanoparticles are used, e.g., prepared from HAuCl₄. Nanoparticles can be any shape and can range in size from about 1 nm to about 10 nm in size, e.g., about 2 nm to about 8 nm, about 4 to about 6 nm, or about 5 nm in size. Methods for making colloidal metal nanoparticles, including gold colloidal nanoparticles from HAuCl.sub.4, are known to those having ordinary skill in the art. For example, the methods described herein as well as those described elsewhere (e.g., US 2001/005581; 2003/0118657; and 2003/0053983) are useful guidance to make nanoparticles.

In certain cases, a nanoparticle can have two, three, four, five, six, or more immunogenic compositions linked to its surface. Typically, many molecules of an immunogenic composition are linked to the surface of the nanoparticle at many locations. Accordingly, when a nanoparticle is described as having, for example, two immunogenic compositions linked to it, the nanoparticle has two distinct immunogenic compositions, each having its own unique molecular structure, linked to its surface. In some cases, one molecule of an immunogenic composition can be linked to the nanoparticle via a single attachment site or via multiple attachment sites.

An immunogenic composition can be linked directly or indirectly to a nanoparticle surface. For example, linked directly to the surface of a nanoparticle or indirectly through an intervening linker.

Any type of molecule can be used as a linker. For example, a linker can be an aliphatic chain including at least two carbon atoms (e.g., 3, 4, 5, 6, 7, 8, 9, 10 or more carbon atoms), and can be substituted with one or more functional groups including ketone, ether, ester, amide, alcohol, amine, urea, thiourea, sulfoxide, sulfone, sulfonamide, and disulfide functionalities. In cases where the nanoparticle includes gold, a linker can be any thiol-containing molecule. Reaction of a thiol group with the gold results in a covalent sulfide (—S—) bond. Linker design and synthesis are well known in the art.

Any type of immunogenic composition or any type of additional agent can be linked to a nanoparticle. For example, an additional agent can be a therapeutic agent that has a therapeutic effect in the body. Examples of therapeutic agents include, without limitation, anti-angiogenic agents, anti-inflammatory agents, anti-bacterial agents, anti-fungal agents, growth factors, immunostimulatory agents. A therapeutic agent can be in any physical or chemical form, including an antibody, an antibody fragment, a receptor, a receptor fragment, a small-molecule, a peptide, a nucleic acid, and a peptide-nucleic acid.

A therapeutic agent can function as a targeting agent in addition to functioning as a therapeutic agent. A targeting functionality can allow nanoparticles to accumulate at the target at higher concentrations than in other tissues. In general, a targeting molecule can be one member of a binding pair that exhibits affinity and specificity for a second member of a binding pair. For example, an antibody or antibody fragment therapeutic agent can target a nanoparticle to a particular region or molecule of the body (e.g., the region or molecule for which the antibody is specific) while also performing a therapeutic function. In some cases, a receptor or receptor fragment can target a nanoparticle to a particular region of the body, e.g., the location of its binding pair member. Other therapeutic agents such as small molecules can similarly target a nanoparticle to a receptor, protein, or other binding site having affinity for the therapeutic agent.

The formulations of the present embodiment may also include other agents useful for pH maintenance, solution stabilization, or for the regulation of osmotic pressure. Examples of the agents include but are not limited to salts, such as sodium chloride, or potassium chloride, and carbohydrates, such as glucose, galactose or mannose, and the like.

The immunogenic composition should be administered to the patient in an amount effective to stimulate a protective immune response in the patient. For example, the immunogenic composition may be administered to humans in one or more doses, each dose containing is 10³ to 10¹¹ PFU, for example 10² or 10³ or 10⁴ or 10⁵ or 10⁶, more preferably 10³ to 10⁹ or 10¹⁰ or 10¹¹ PFU .

The immunogenic compositions as discussed herein can also be combined with at least one conventional vaccine (e.g., inactivated, live attenuated, or subunit) directed against the same pathogen or at least one other pathogen of the species to which the composition or vaccine is directed.

Certain subjects can be identified as suited for administration of the immunogenic compositions of the invention. In certain preferred embodiments, the subjects would receive an immunogenic composition comprising a vector prime.

For example, infants are suited to receive immunogenic compositions consisting of a vector prime boost of, for example, a first dose at birth and a second dose at, for example, 1 mo of age or any period of time thereafter. The elderly or immunocompromised are another population that can be identified as subjects that can be administered an immunogenic composition consisting of a vector prime boost, as described herein.

### Prime Boosting

The prime-boost regimen according to the invention can be used in animals of any age, advantageously young animals (e.g., animals that have detectable maternal antibodies and/or are suckling or nursing or breast-feeding), pre-adult animals (animals that are older than being a young animal but have not yet reached maturity or adulthood or an age to mate or reproduce), adult animals (e.g., animals that are of an age to mate or reproduce or are beyond such a period in life), and it is advantageous to employ the prime-boost regimen in pregnant females or females prior to giving birth, laying, or insemination. The prime boost regimen may be a homologous prime boost (e.g. the same immunogenic composition is administered as the prime and the boost) or a heterologous prime boost (e.g. different immunogenic compositions are administered as the prime and the boost).

The term “vector priming” is meant to refer to the delivery of a gene encoding a vaccine antigen (or the immunogenic composition) by way of an expression vector. In certain embodiments, it means that the vector-based gene delivery will be a first exposure to the immunogenic composition, followed by one or more subsequent “booster” dose or doses of immunogenic compositions. The priming administration (priming) is the administration of a immunogenic or immunological composition type and may comprise one, two or more administrations. The boost administration is the administration of a second immunogenic or immunological composition type and may comprise one, two or more administrations, and, for instance, may comprise or consist essentially of annual administrations. The “boost” may be administered anytime after the priming, for example in certain embodiments from about 2 weeks to about 12 months after the priming, such as from about 6 week to about 6 months, or from about 3 to about 6 weeks after the priming, or from about 4 weeks after the priming.

The prime-boost regimen is especially advantageous to practice in a young animal, as it allows vaccination or immunization at an early age, for instance, the first administration in the prime-boost regimen when practiced on a young animal can be at an age at which the young animal has maternal antibodies. Another advantage of this regimen is that it can provide a degree of safety for pregnant females present in the same location or in close proximity to the young or to each other. Thus, the invention provides a prime-boost immunization or vaccination method against, for example, an a disease or infection, and the method may be practiced upon a young animal, wherein the priming is done at a time that the young animal has maternal antibodies against the disease or infection to be treated, with the boost advantageously at a time when maternal antibodies may be waning or decreasing or normally not present, such as a period of time post-breastfeeding.

The amounts (doses) administered in the priming and the boost and the route of administration for the priming and boost can be as herein discussed, such that from this disclosure and the knowledge in the art, the prime-boost regimen can be practiced without undue experimentation. Furthermore, from the disclosure herein and the knowledge in the art, the skilled artisan can practice the methods, kits, etc. herein with respect to any of the herein-mentioned target species.

In certain preferred embodiments, the immunogenic composition is administered in a prime boost regimen. The prime boost regimen can be a homologous prime boost or the prime boost regimen can be a heterologous prime and boost.

In certain examples, the prime is delivered by mucosal administration. In other examples, the boost is delivered by parental administration. For example, HPV vector can be used as a mucosal prime to augment a subsequent boost with a parenterally delivered immunogenic composition.

### Epithelial Disruption

### Epithelium

Epithelium refers to cells that line hollow organs and glands and those that make up the outer surface of the body. Epithelial cells are arranged in single or multiple layers, depending on the organ and location, and epithelia are classified into types on the basis of the number of layers deep and the shape of the superficial cells. Epithelium lines both the outside and the inside cavities and lumen of bodies. The outermost layer of our skin is composed of stratified squamous, keratinized epithelial cells. Mucous membranes lining the inside of the mouth, the oesophagus, and part of the cervicovaginal tract and rectum are lined by nonkeratinized stratified squamous epithelium. Other, open to outside body cavities are lined by simple squamous or columnar epithelial cells. Other epithelial cells line the insides of the lungs, the gastrointestinal tract, the reproductive and urinary tracts, and make up the exocrine and endocrine glands. The outer surface of the cornea is covered with fast-growing, epithelial cells that are easily regenerated. Endothelium, which comprises the inner lining of blood vessels, the heart, and lymphatic vessels, is a specialized form of epithelium. Another type, Mesothelium, forms the walls of the pericardium, pleurae, and peritoneum.

Epithelial surfaces according to the invention, in certain examples can be, but are not limited to, cervicovaginal, oral, nasal, penile, anal, epidermal and respiratory surfaces.

Epithelial disruption can be carried out by a number of means. In certain examples, epithelial disruption can be carried out by chemical means. In other certain embodiments, epithelial disruption can be carried out by physical means. One of skill in the art will easily recognize that any chemical or physical means to disrupt an epithelial surface can be used in the methods of the invention as described herein.

In certain embodiments of the invention, the one or more agents or treatments to disrupt an epithelial surface are administered prior to administration of the immunogenic composition.

By disrupt an epithelial surface is meant to compromise the barrier function of the epithelium. The epithelium provides a barrier to the underlying layers or cells. In certain examples, disrupting the epithelium allows the immunogenic composition to access the lateral or basolateral surface of cells, and/or the basement membrane that separates the epithelium from the underlying dermis.

In certain examples, one or more chemical agents may be used to disrupt an epithelial surface. A chemical agent can be any caustic agent, e.g. an acid. When the agent to disrupt the epithelial surface is a chemical agent, it may be selected from but not limited to, a detergent, an acid and an antibody treatment.

In a clinical setting, one of skill in the art will easily understand that an appropriate concentration or dosage of the agent will need to be used in order to disrupt the epithelium without causing undue harm to the subject.

In certain cases, the chemical agent is a detergent may be an ionic or a non-ionic detergent. Examples of non-ionic detergents include Brj-35, n-Dodecyl-β-D-Maltoside, Octyl β-Glucoside. Examples of ionic detergents include Sodium Cholate and Sodium deoxycholate. A review of detergents is provided in Neugebauer, J. M., Detergents: an overview. Methods Enzymol, 182, 239-253 (1990), incorporated by reference in its entirety herein.

In certain preferred examples, the detergent is nonoxynol-9 (N-9), a non-ionic nonoxynol surfactant that is used as an ingredient in various cleaning and cosmetic products, but is also widely used in contraceptives for its spermicidal properties. The structure of N-9 is shown below. In certain embodiments, derivatives of N-9 may be used.

In other examples, the one or more treatments to disrupt the epithelial surface is a physical treatment. In certain preferred example, the physical treatment is selected from, but not limited to, abrasion, adhesion, needle puncture, temperature treatment, electrical treatment, sonication, and radiative treatment.

An abrasion may be a scratch or wound that is, in certain embodiments, mechanically created or manually created.

In certain embodiments, a physical treatment to disrupt an epithelial surface may be carried out with a laser.

In certain cases, the physical treatment is carried out with a device or a tool.

It is easily envisioned that a combination of treatments may be used in the methods of the invention, e.g. one or more chemical treatments in combination with one or more physical treatments.

### Kits

The present compositions may be assembled into kits or pharmaceutical systems for use in eliciting an immune response in a subject.

In certain preferred embodiments, the kits can be used in methods of eliciting an immune response in a subject. Preferably, the kits will comprise a viral capsid, e.g., a papillomavirus or adenovirus capsid, wherein the capsid comprises L1 and L2 proteins, and wherein the capsid contains a vector comprising one or more nucleic acid segments, or fragments thereof, a pharmaceutically acceptable carrier, and instructions for use in administration to a disrupted epithelial surface.

The kit may further contain a chemical or mechanical instrument for disrupting the epithelium.

Kits according to this aspect of the invention comprise a carrier means, such as a box, carton, tube or the like, having in close confinement therein one or more container means, such as vials, tubes, ampules, bottles and the like. The kits of the invention may also comprise associated instructions for using the compounds of the invention for use in eliciting an immune response capable of preventing a viral infection in a subject. The kits may also comprise instructions for using the compounds of the invention in administration to a disrupted epithelial surface, as described herein.

Kits or pharmaceutical systems according to the invention described herein may further contain an adjuvant. Adjuvants can be selected from, but are not limited to, oil emulsions, mineral compounds, bacterial products, liposomes, and immunostimulating complexes. Examples of adjuvants contained in the kits include, but are not limited to, aluminum salts, oil-in-water emulsion formulations (with or without other specific immunostimulating agents such as muramyl peptides or bacterial cell wall components, such as for example (a) MF59 (PCT Publ. No. WO 90/14837), containing 5% Squalene, 0.5% Tween 80, and 0.5% Span 85 (optionally containing various amounts of MTP-PE (see below), although not required) formulated into submicron particles using a microfluidizer such as Model 110y microfluidizer (Microfluidics, Newton, Mass.), (b) SAF, containing 10% Squalane, 0.4% Tween 80, 5% pluronic-blocked polymer L121, and thr-MDP (see below) either micro fluidized into a submicron emulsion or vortexed to generate a larger particle size emulsion, and (c) RIBI.TM adjuvant system (RAS), (Ribi Immunochem, Hamilton, Mo.) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (DETOX™); (3) saponin adjuvants, such as STIMULON™ (Cambridge Bioscience, Worcester, Mass.) may be used or particles generated therefrom such as ISCOMs (immunostimulating complexes); (4) Complete Freunds Adjuvant (CFA) and Incomplete Freunds Adjuvant (IFA); (5) cytokines, such as interleukins (e.g., IL-1, IL-2, IL-4, IL-5, IL-6, IL-7, IL-12, etc.), interferons (e.g., gamma interferon), macrophage colony stimulating factor (M-CSF), tumor necrosis factor (TNF), etc; and (6) other substances that act as immunostimulating agents to enhance the effectiveness of the composition. Examples include Alum, and MF59, interferon alpha, Klebsiella pneumoniae glycoprotein and interleukin-2, and chitosans.

easily regenerated. Endothelium, which comprises the inner lining of blood vessels, the heart, and lymphatic vessels, is a specialized form of epithelium. Another type, Mesothelium, forms the walls of the pericardium, pleurae, and peritoneum.

Epithelial surfaces according to the invention, in certain examples can be, but are not limited to, cervicovaginal, oral, nasal, penile, anal, epidermal and respiratory surfaces.

Epithelial disruption can be carried out by a number of means. In certain examples, epithelial disruption can be carried out by chemical means. In other certain embodiments, epithelial disruption can be carried out by mechanical, e.g., physical, means. One of skill in the art will easily recognize that any chemical or mechanical means to disrupt an epithelial surface can be used in the methods of the invention as described herein.

In certain embodiments of the invention, the one or more agents or treatments to disrupt an epithelial surface are administered prior to administration of the immunogenic composition.

By disrupt an epithelial surface is meant to compromise the barrier function of the epithelium. The epithelium provides a barrier to the underlying layers or cells. In certain examples, disrupting the epithelium allows the immunogenic composition to access the lateral or basolateral surface of cells, and/or the basement membrane that separates the epithelium from the underlying dermis.

In certain examples, one or more chemical agents may be used to disrupt an epithelial surface. When the agent to disrupt the epithelial surface is a chemical agent, it may be selected from but not limited to, a detergent, an acid and an antibody treatment.

In a clinical setting, one of skill in the art will easily understand that an appropriate concentration or dosage of the agent will need to be used in order to disrupt the epithelium without causing undue harm to the subject.

In certain cases, the chemical agent is a detergent may be an ionic or a non-ionic detergent. Examples of non-ionic detergents include Brj-35, n-Dodecyl-β-D-Maltoside, Octyl β-Glucoside. Examples of ionic detergents include Sodium Cholate and Sodium deoxycholate. A review of detergents is provided in Neugebauer, J. M., Detergents: an overview. Methods Enzymol, 182, 239-253 (1990), incorporated by reference in its entirety herein.

In certain preferred examples, the detergent is nonoxynol-9 (N-9), a non-ionic nonoxynol surfactant that is used as an ingredient in various cleaning and cosmetic products, but is also widely used in contraceptives for its spermicidal properties. The structure of N-9 is shown below. In certain embodiments, derivatives of N-9 may be used.

In other examples, the one or more treatments to disrupt the epithelial surface is a mechanical, i.e., physical treatment. In certain preferred example, the mechanical treatment is selected from, but not limited to, abrasion, adhesion, temperature treatment, electrical treatment, sonication, and radiative treatment.

An abrasion may be a scratch or wound that is, in certain embodiments, mechanically created or manually created.

In certain embodiments, a physical treatment to disrupt an epithelial surface may be carried out with a laser.

In certain cases, the physical treatment is carried out with a device or a tool.

It is easily envisioned that a combination of treatments may be used in the methods of the invention, e.g. one or more chemical treatments in combination with one or more physical treatments.

Having now generally described the invention, the same will be more readily understood through reference to the following Examples, which are provided by way of illustration, and are not intended to be limiting of the present invention, unless specified.

### EXAMPLES

### Example 1

Under healthy conditions, the stratified squamous epithelia that line mucosal surfaces are thought to provide a barrier against the infectious entry of a wide variety of microorganisms. Techniques for overcoming the barrier function of the genital epithelium in female mice have recently been described (Roberts et al. (2007) Nat. Med. 13:857, incorporated by reference in its entirety herein). In that report, it was shown that physical abrasion of the female genital tract permitted infectious entry of human papillomavirus (HPV)-based gene delivery vectors into keratinocytes that form the lining the genital tract. Over-the-counter spermicides containing the detergent nonoxonol-9 (N-9) were also highly effective at disrupting mucosal barrier function and potentiating HPV infection.

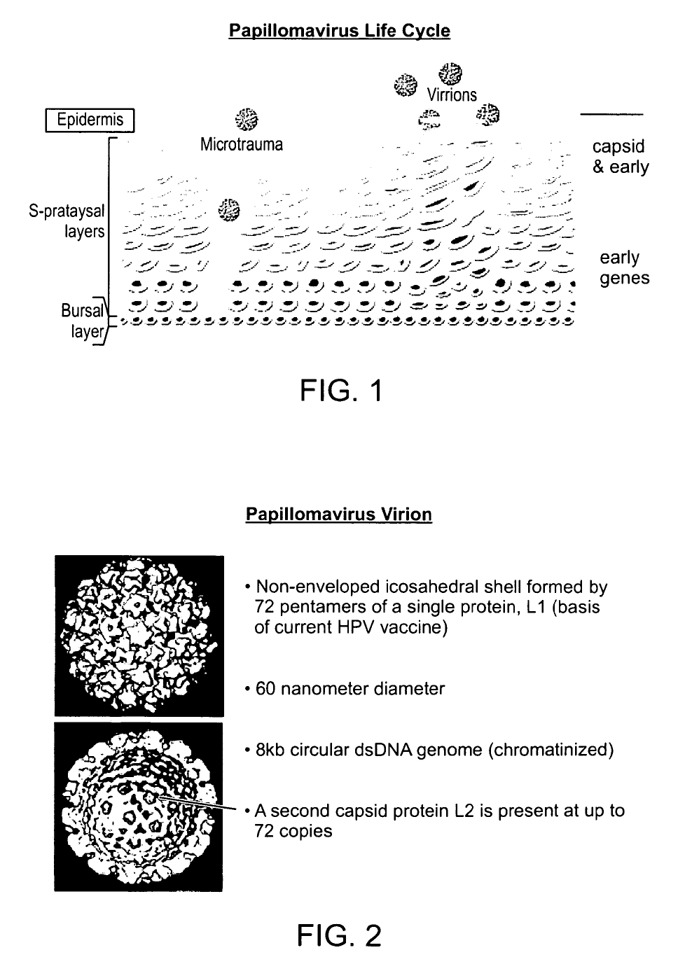

Papillomaviruses infect epithelial cells, and are thought to target basal keratinocytes or keratinocyte stem cells resident in the bottom layers of the epithelium. The life cycle of human papillomaviruses is tightly linked to the differentiation program of keratinocytes in the stratified epithelium. The papillomavirus lifecycle is shown in a schematic in FIG. 1. The papillomavirus virion is shown in FIG. 2. Papillomavirus particles are comprised of the products of the L1 (Major capsid protein) and L2 (Minor capsid protein) genes. L1 can spontaneously self-assemble into a 60 nanometer, 72-pentamer icosahedral structure that closely resembles authentic papillomavirus virions. Previously, a vaccine for HPV has been developed based on the self-assembly of the L1 protein into virus-like particles. The capsid protein L2 is present in authentic virions at up to 72 copies. HPV virions are produced in the upper strata of infected epithelium. It has been shown that when L1 and L2 are co-expressed in cultured mammalian cells, they can self-assemble and take up plasmid DNA present in the cell nucleus, provided the plasmid is less than 8 kb in size. The resulting L1/L2 pseudovirions are competent for transducing the encapsidated plasmid DNA into a variety of cell types in vitro or into keratinocytes in the murine genital tract (Roberts et al. (2007) Nat. Med. 13:857, as above and incorporated by reference in its entirety herein; also Buck, C. B. et al. J. Virol. 78:751-757, 2004, incorporated by reference in its entirety herein).

A 2007 review, (Buck C. B. et al, Current Protocols in Cell Biology 26.1.1-26.1.19, December 2007, incorporated by reference in its entirety herein), outlines the production and propagative amplification of papillomaviral vectors.

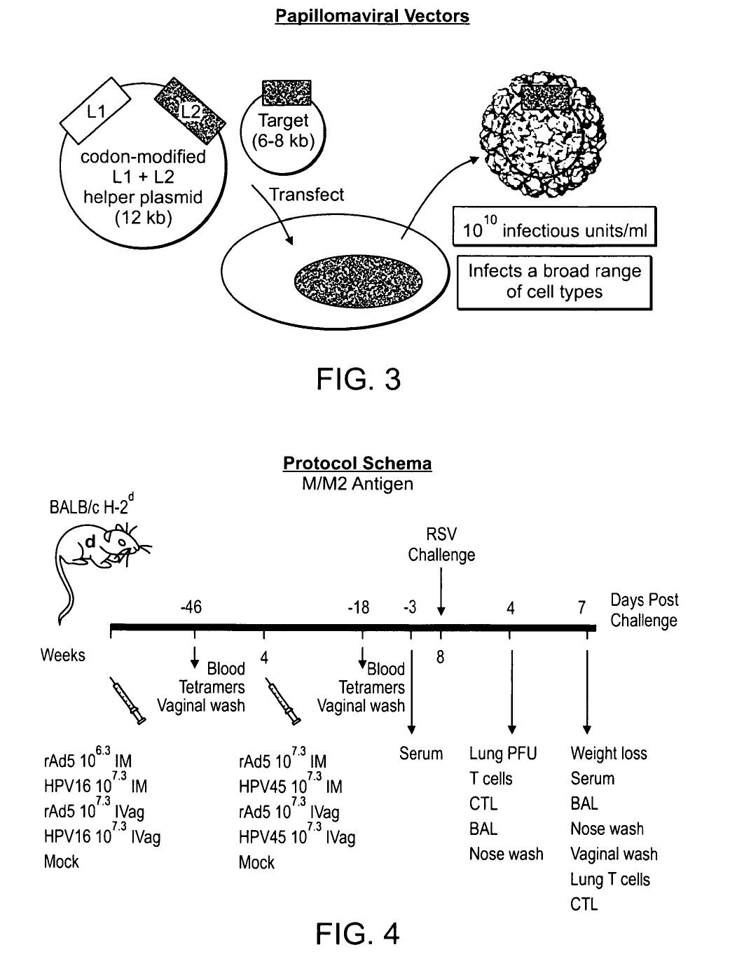

HPV type 16 and type 45 vectors were generated that carry a model immunogen consisting of the fused M and M2 genes (M/M2) of respiratory syncytial virus (Rutigliano et al. (2007) Virology 362:314—attached and U.S. Provisional Application No. 60/872,071, incorporated by reference in its entirety herein and related PCT application entitled CODON MODIFIED IMMUNOGENIC COMPOSITIONS AND METHODS OF USE filed 30 Nov., 2007, Application No. not yet assigned, incorporated by reference in its entirety herein). A schematic of papilloma viral vectors is shown in FIG. 3.

In a first set of experiments, the immunogenicity of DNA delivered by papillomaviral vectors versus gene delivery by a recombinant adenoviral vector (rAdS) was evaluated. The experiments are outlined in the protocol schema in FIG. 4. BALB/c mice were first immunized with replication-defective recombinant adenovirus serotype 5 (rAd5) expressing the M/M2 gene or HPV16 papillomaviral vectors containing DNA expressing the M/M2 gene. Immunization was delivered either intramuscularly (IM) or intravaginally (IVag). Prior to IVag delivery mice were treated with depoprovera and intravaginal nonoxynol-9. Secondary immunization was given 4 weeks later as a homologous boost with rAdS-M/M2 or with a heterologous HPV45 papillomaviral vector containing the M/M2 plasmid (HPV45-M/M2). A control group received mock vectors given both IM and IVag. Readouts for immunogenicity included measures of cell-mediated and humoral immunity before and after challenge with RSV administered 4 weeks after the last immunization. Weight loss and quantitation of RSV in lung was performed post challenge.

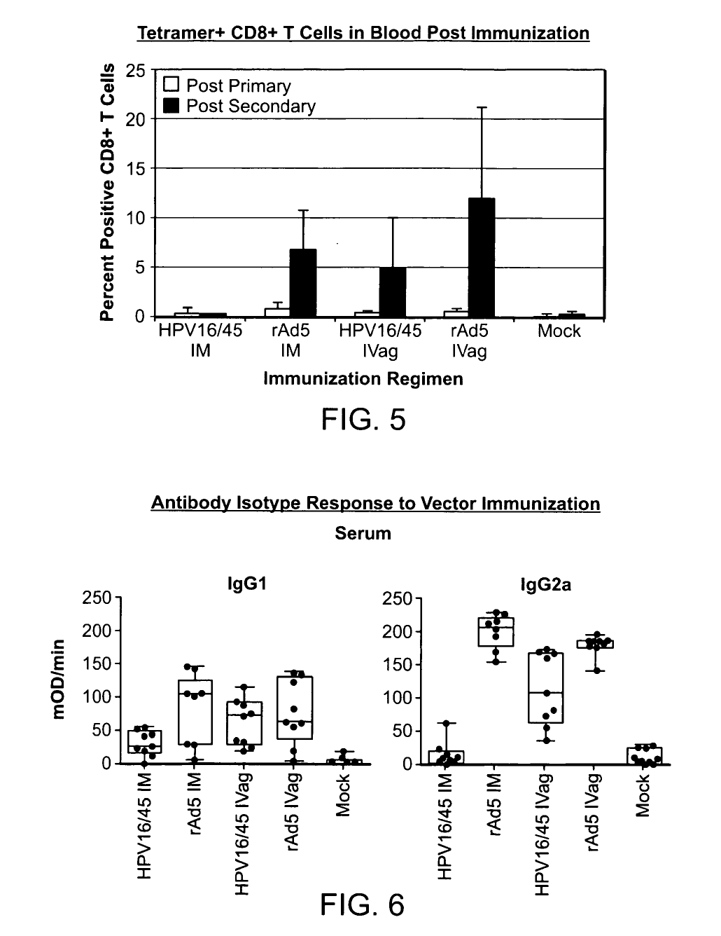

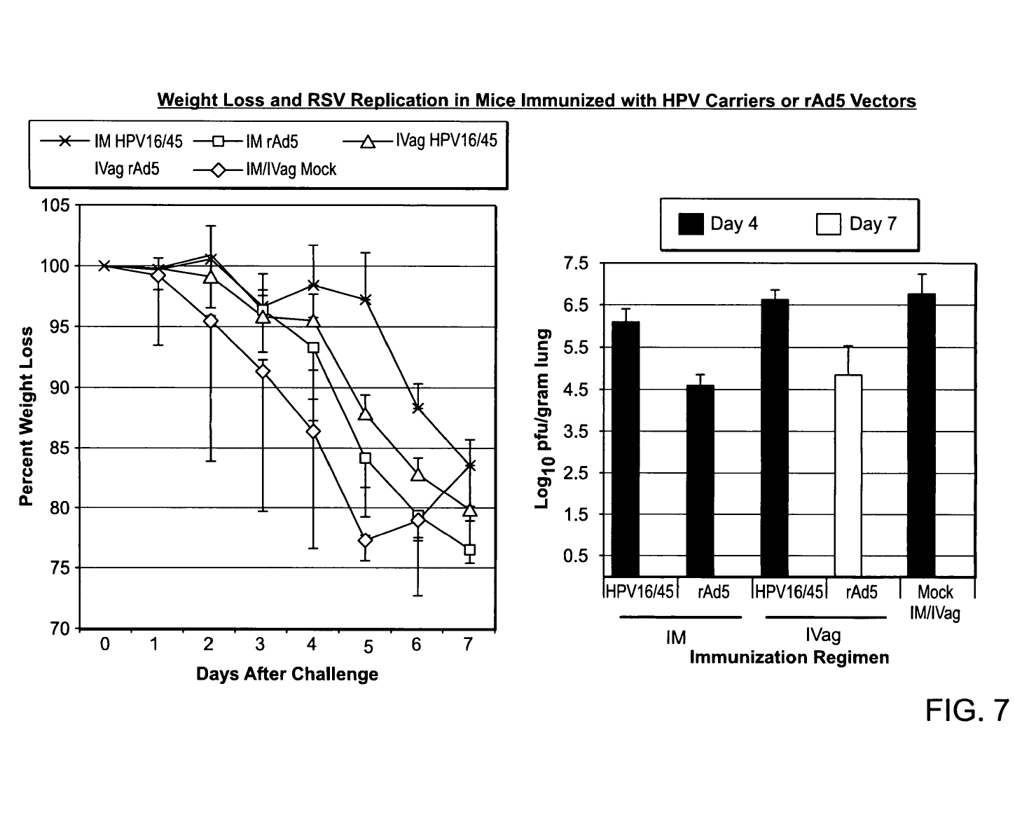

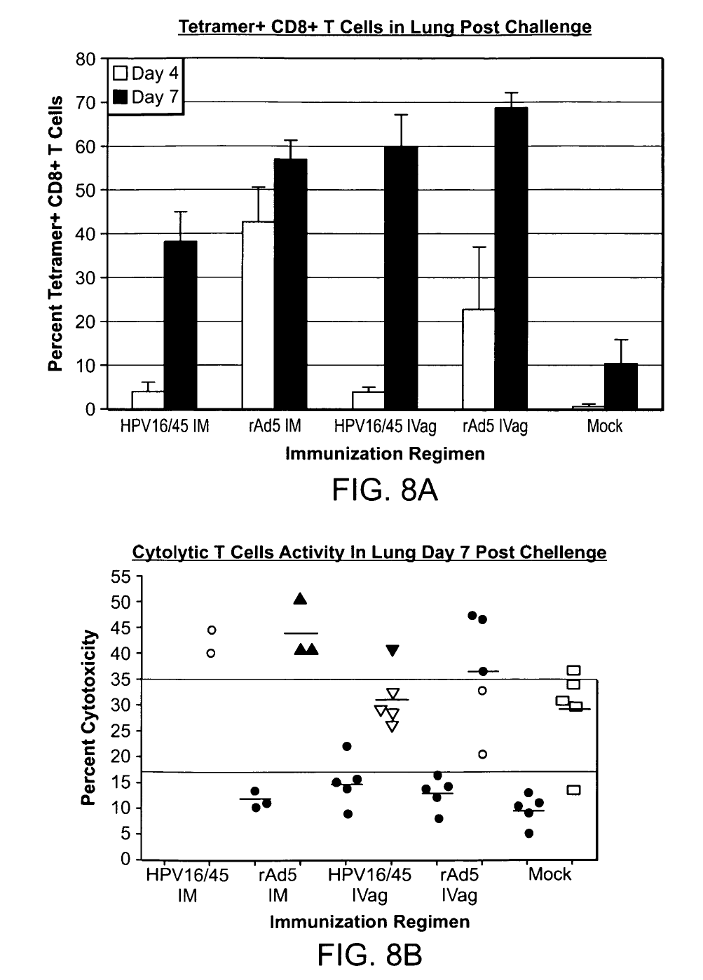

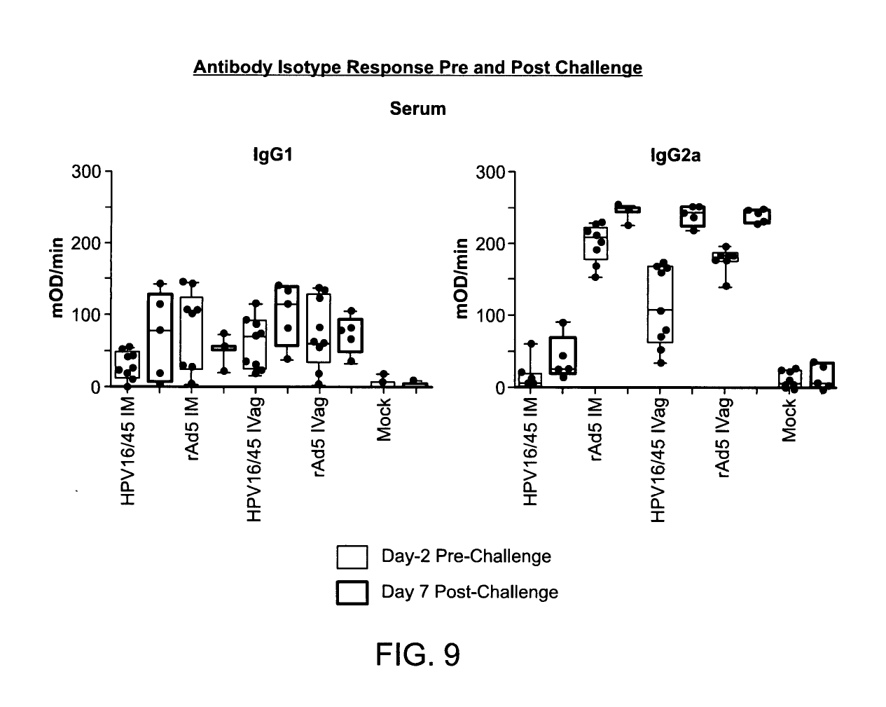

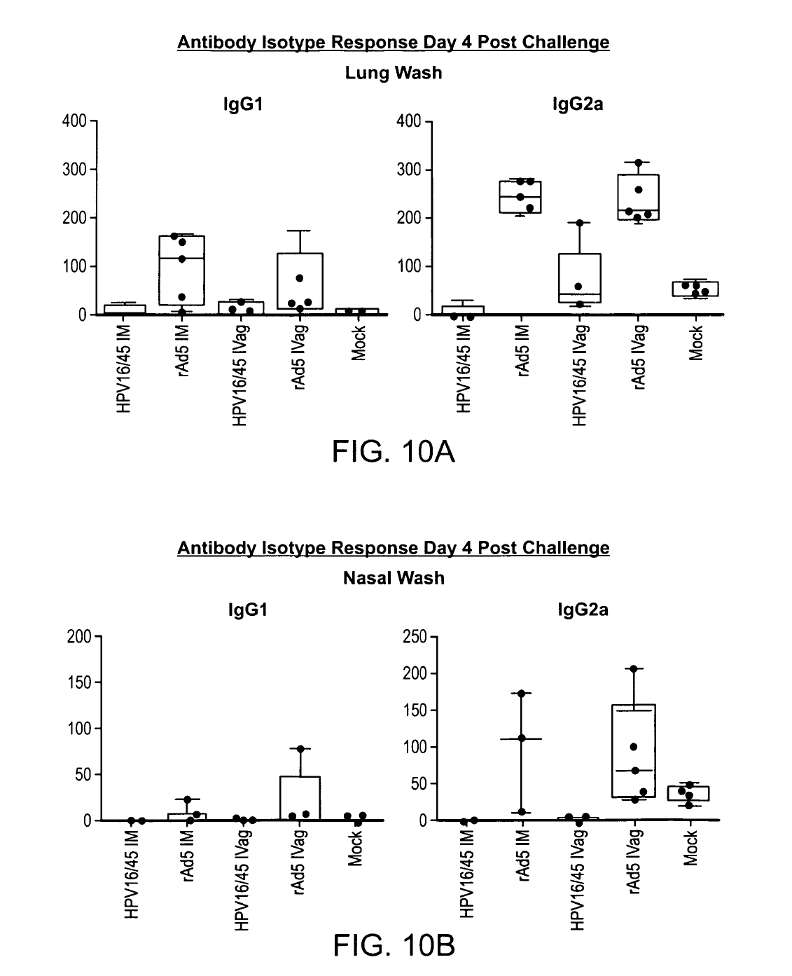

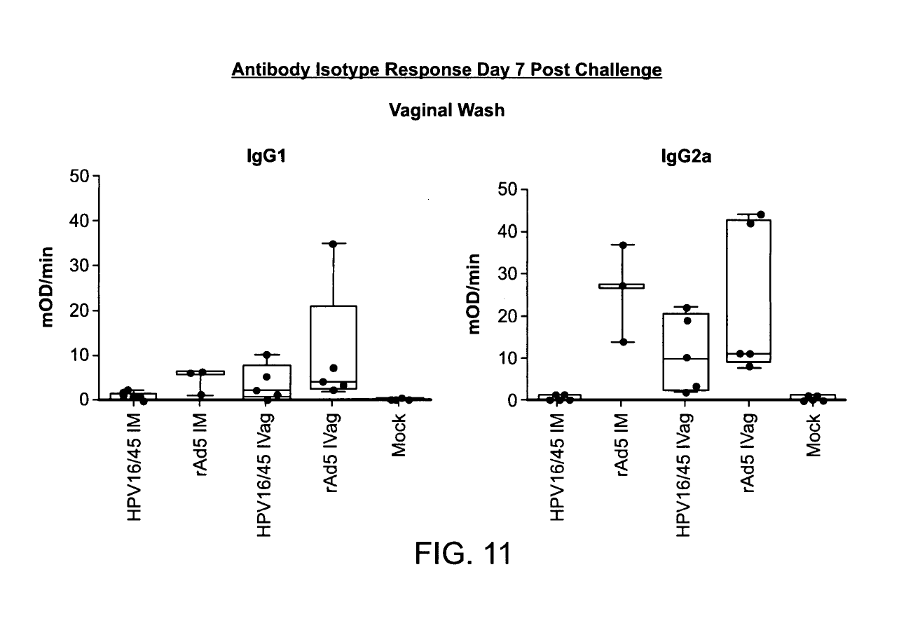

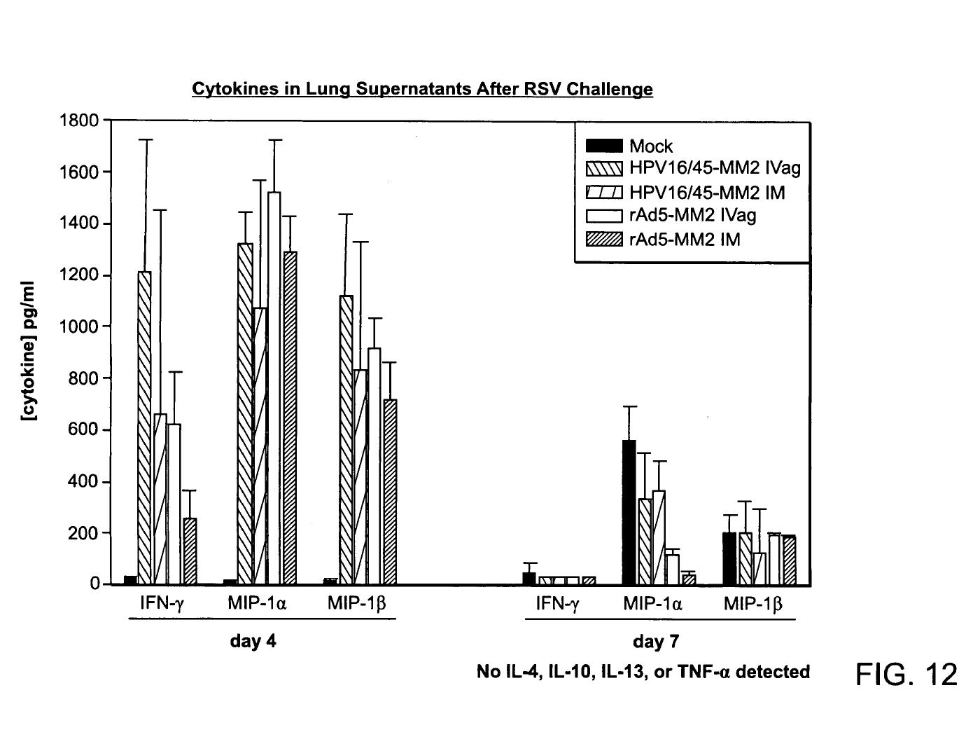

The results are shown in FIGS. 5-12. Post secondary immunization, CD8+ T cell responses specific for the M2 epitope were detected in blood by tetramer analysis in both groups receiving rAd5, but also in the group immunized with HPV IVag (FIG. 5). Likewise, both the rAd5 and HPV IVag elicited M/M2-specific antibody responses detected in serum by ELISA prior to RSV challenge (FIG. 6). The antibody isotype was predominantly IgG2a suggesting that vaccination induced a Th1-type immune response with dominant IFN-γ production. The M/M2 antigen has 3 well characterized CD8+ T cell epitopes and at least 2 CD4+ T cell epitopes. The M/M2 antigen can also elicit an antibody response, but since M and M2 are virion core antigens, antibodies against them cannot neutralize RSV. Only the F and G glycoproteins present on the surface of RSV are targets for neutralizing antibodies. Therefore the M/M2 is an experimental antigen used to evaluate the value of gene-based vector delivery approaches for inducing T cell and antibody responses, but is not expected to produce a protective immune response or to prevent infection with RSV that would only occur if F or G were contained in the vaccine antigen. After challenge with RSV, mice immunized with rAd5 or HPV vectors exhibited earlier weight loss than the mock-immunized group, which is a reflection of the earlier T cell response (FIG. 7). There was also evidence of earlier recovery in the immunized mice, particularly in the rAd5 groups that also had evidence of diminished RSV replication in lungs on day 4 post challenge (FIG. 7). The M2-specific T cell response in lung post challenge measured by tetramer binding was much earlier and more robust in the immunized groups than in the mock-immunized mice, and had good cytolytic activity (FIG. 8). As suggested by the pre-challenge serum antibody responses, the post challenge antibody responses were highest in the HPV vector IVag and rAd5 immunized mice and was primarily the IgG2a isotype (FIG. 9). A similar pattern of M/M2-specific antibody was detected in lung wash and nasal wash (FIG. 10) and in vaginal wash (FIG. 11) post challenge. This pattern is consistent with the cytokines and chemokines detected in lung supernatants. Immunized mice produced large amounts of IFN-γ, MIP-1α, and MIP-1β, and no detectable IL-4, IL-10, IL-13, or TNF-α, all characteristic of a Th1 immune response (FIG. 12).

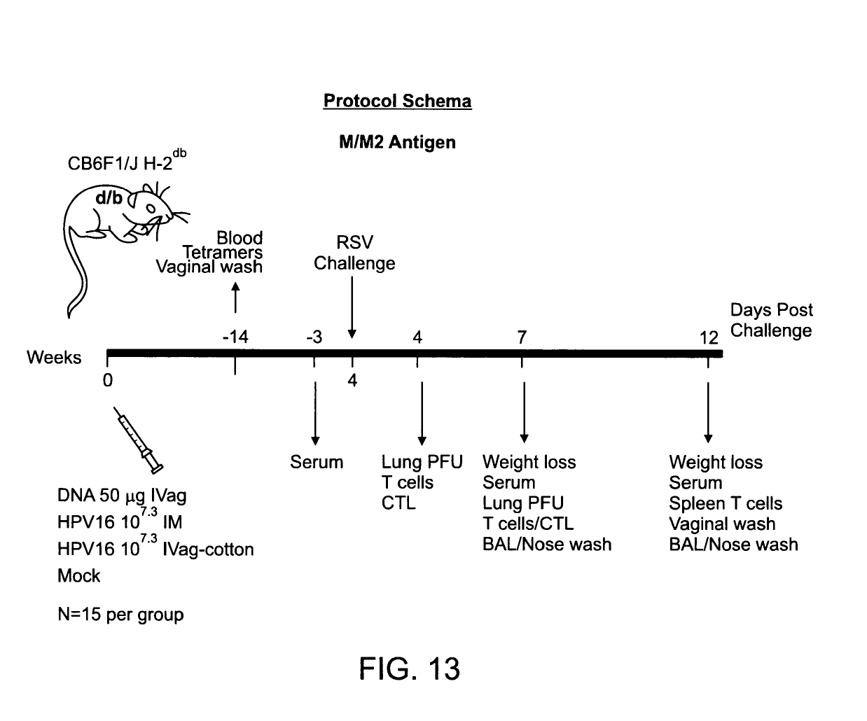

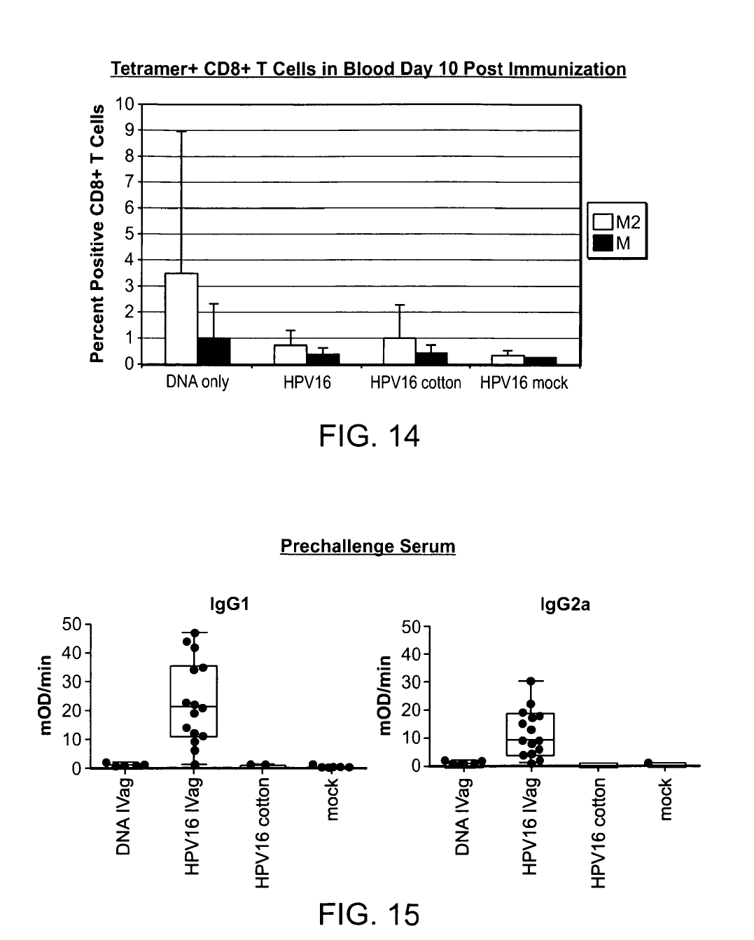

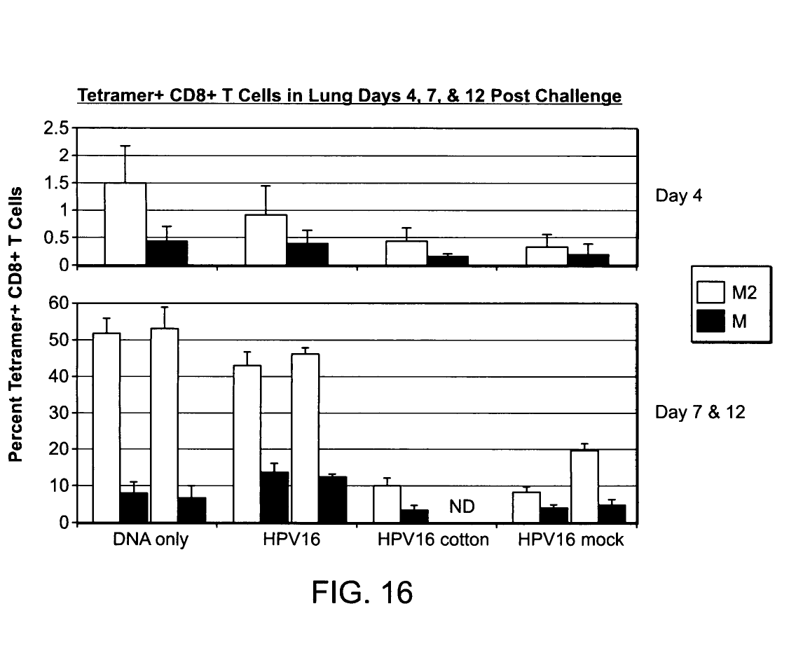

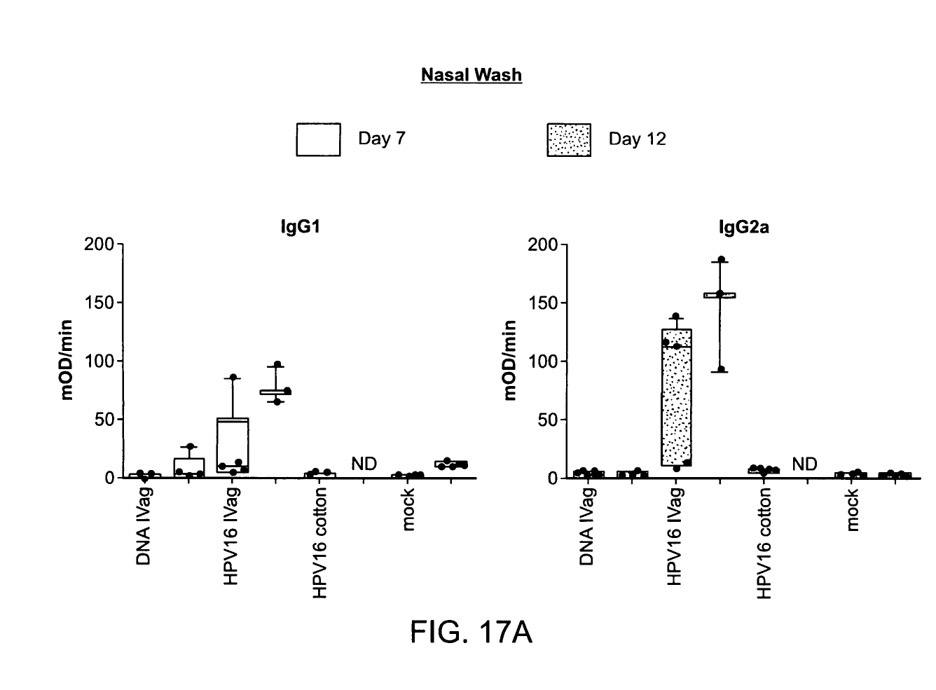

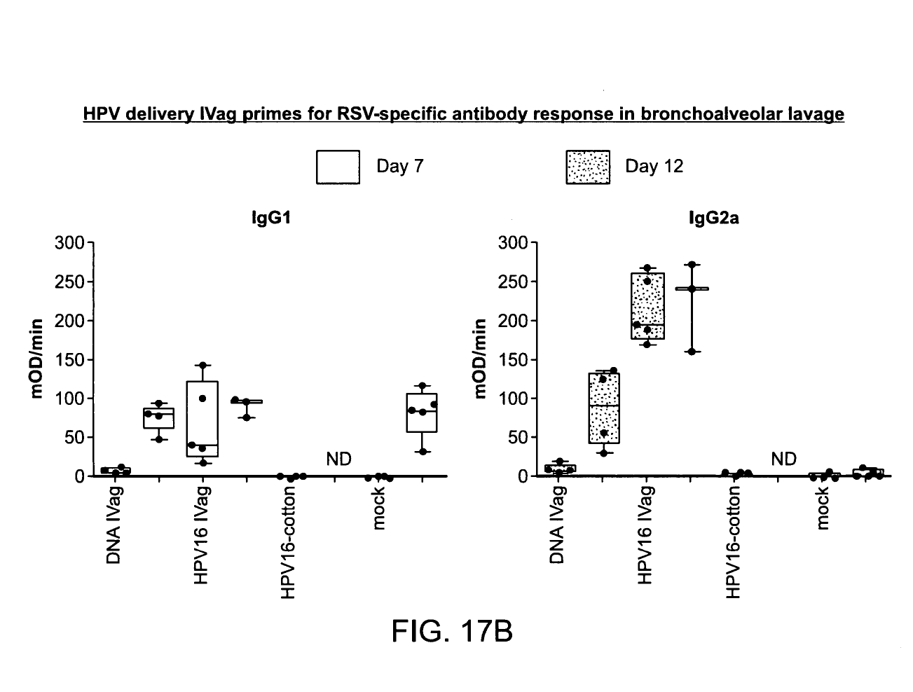

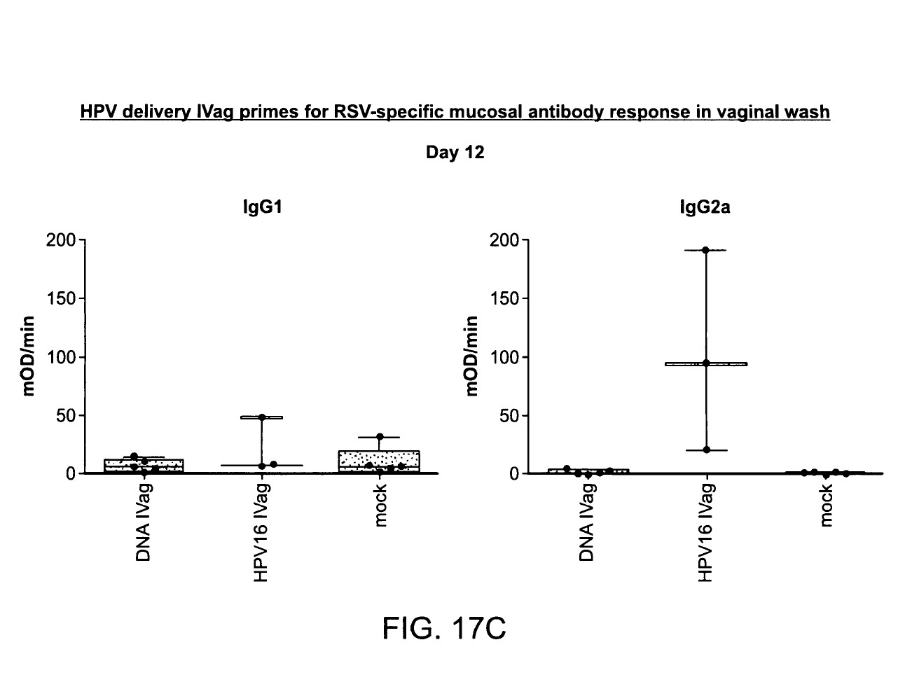

In a second set of experiments, outlined in the protocol schema in FIG. 13, the delivery of either plasmids expressing the M/M2 gene delivered IVag as either naked DNA or contained within HVP16 vectors could be effective given as a single dose to CB6F1/J mice. In addition the HVP16-M/M2 was applied to a cotton pledget and inserted IVag for 4 days. The DNA-M/M2 and HPV16-M/M2 were delivered after pretreatment with depoprovera and N-9, but the group immunized with cotton were not pretreated. Readouts for immunogenicity included measures of cell-mediated and humoral immunity both before and after RSV challenge. A mock immunized control received HPV16 pseudovirus encoding firefly luciferase in place of M/M2. The results are shown in FIGS. 14-17. After a single immunization, M2 and M-specific CD8+ T cells were detected in blood by tetramer staining in immunized mice (FIG. 14), but at a lower level than present after two immunizations (FIG. 5). Serum antibody responses were detected prior to challenge only in the HVP16-M/M2 IVag immunized mice pretreated with depoprovera and N-9. The response was balanced between IgG1 and IgG2a. (FIG. 15). After challenge, the T cell responses measure by tetramer staining showed an earlier, more robust response against both the M and M2 epitopes. The HPV delivered by cotton pledget induced responses no different from mock-immunized mice (FIG. 16). The antibody response was measured in nasal wash post challenge and showed a pattern similar to that seen is prechallenge sera. Only the HPV16-M/M2 IVag immunized mice had a significant antibody response on days 7 and 12 after RSV challenge (FIG. 17). These results show that delivering DNA plasmids expressing a vaccine antigen in a papillomaviral vector can elicit both CD8+ T cell and antibody responses systemically and mucosally with a single immunization when the vaginal epithelium is disrupted.

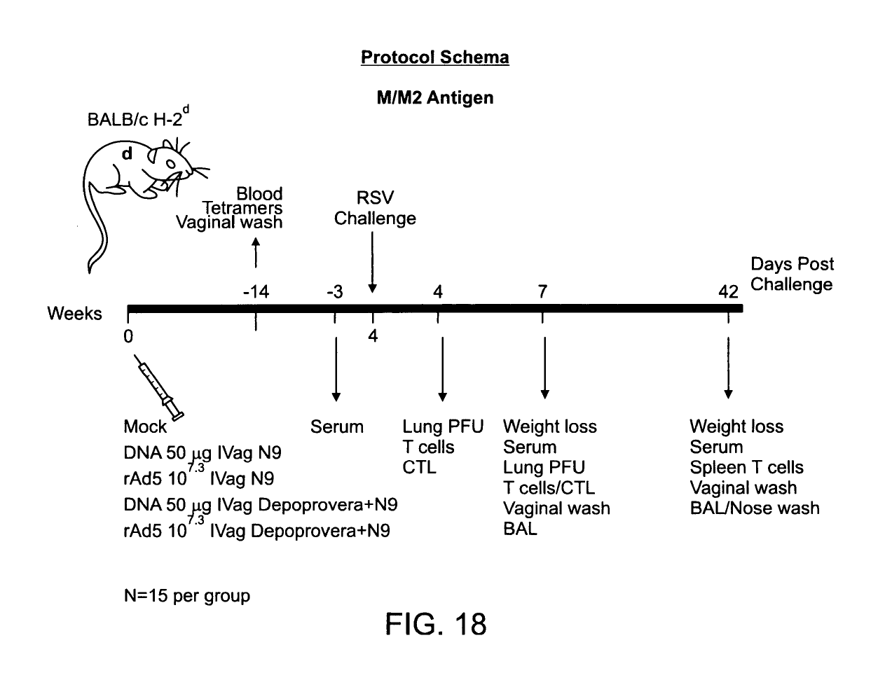

In a third set of experiments, outlined in the protocol schema in FIG. 18, naked DNA or rAd5 were delivered IVag as a single dose to BALB/c mice with either N-9 alone or depoprovera plus N-9 pretreatment. Readouts for immunogenicity included measures of cell-mediated and humoral immunity before and after RSV challenge.

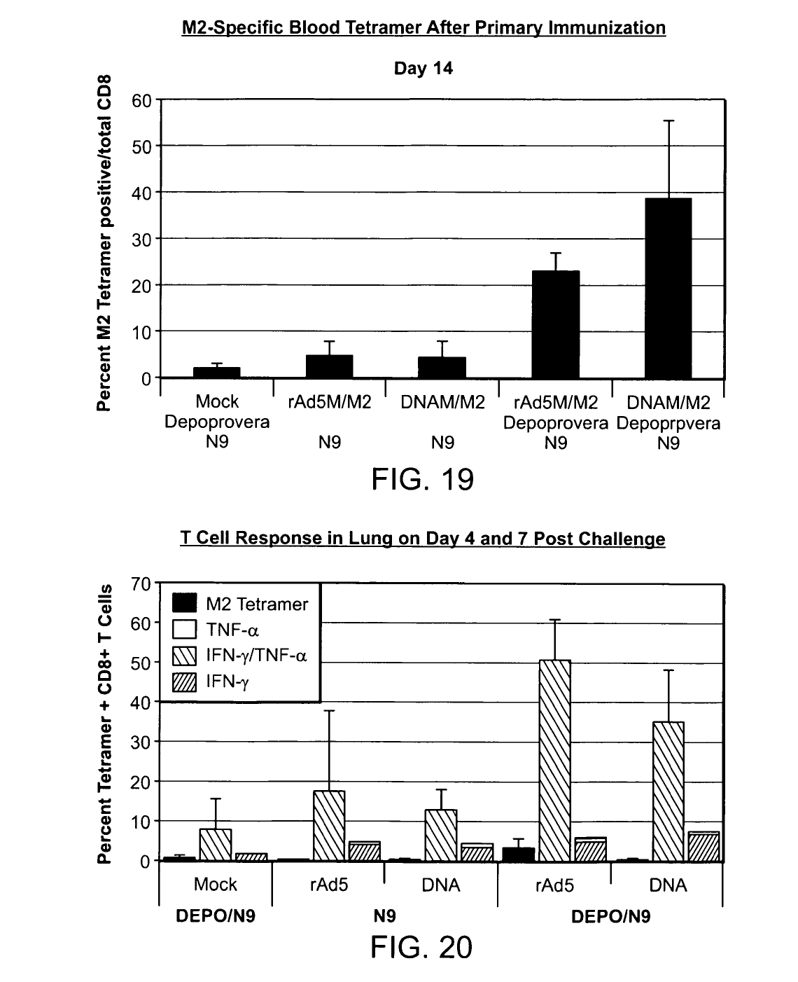

The results for the T cell analysis are shown in FIGS. 19 and 20. Following a single IVag inoculation prior to challenge the depoprovera and N-9 pretreatment group had more robust M2-specific CD8+ T cell responses measured by blood tetramer analysis than mice treated with N-9 alone. However, mice treated with N-9 only had detectable and significant M2-specific CD8+ T cells in blood (FIG. 19). A similar pattern was seen in lung lymphocytes after RSV challenge with depoprovera+N-9 groups having higher responses than N-9 only treated groups, but all immunized mice had greater responses than the mock-immunized group. Mice are known to have a thick and highly cornified vaginal epithelium and optimal approaches for epithelial disruption in other species, including humans, will need to be established.

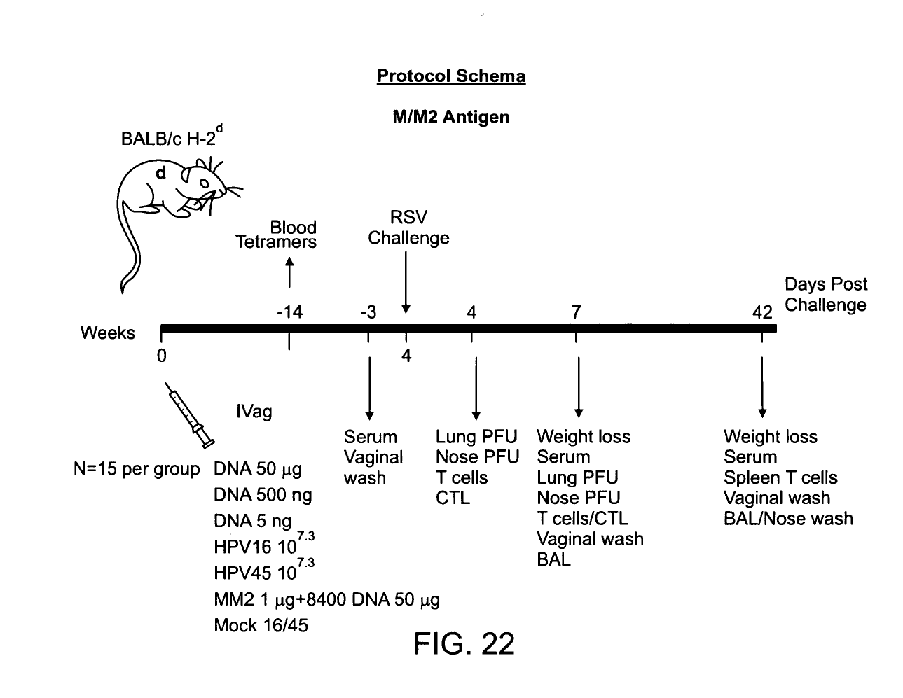

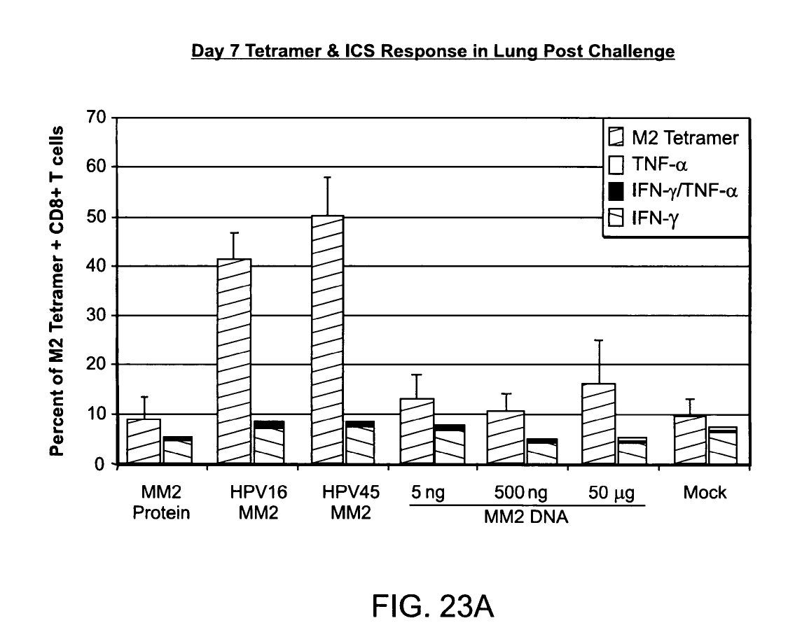

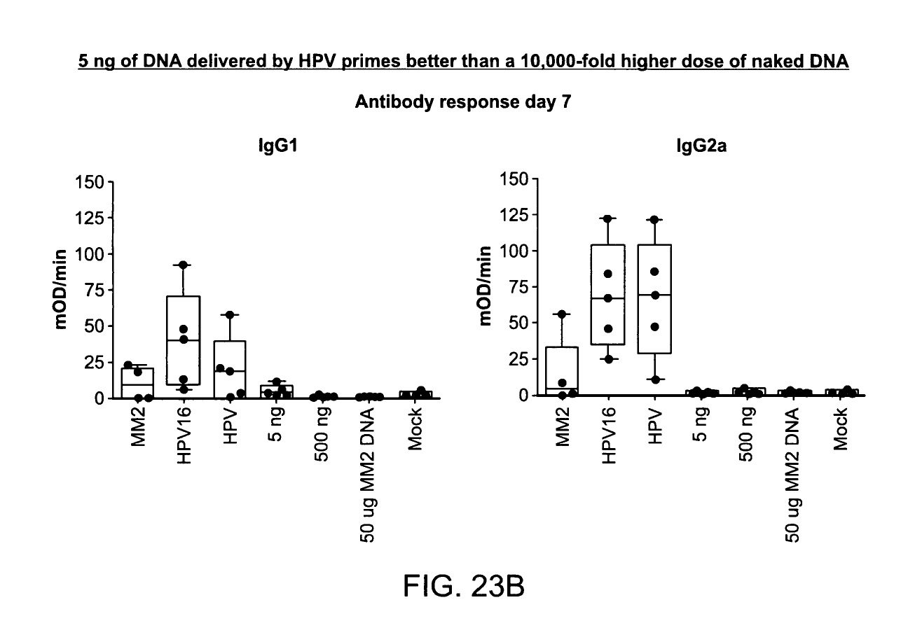

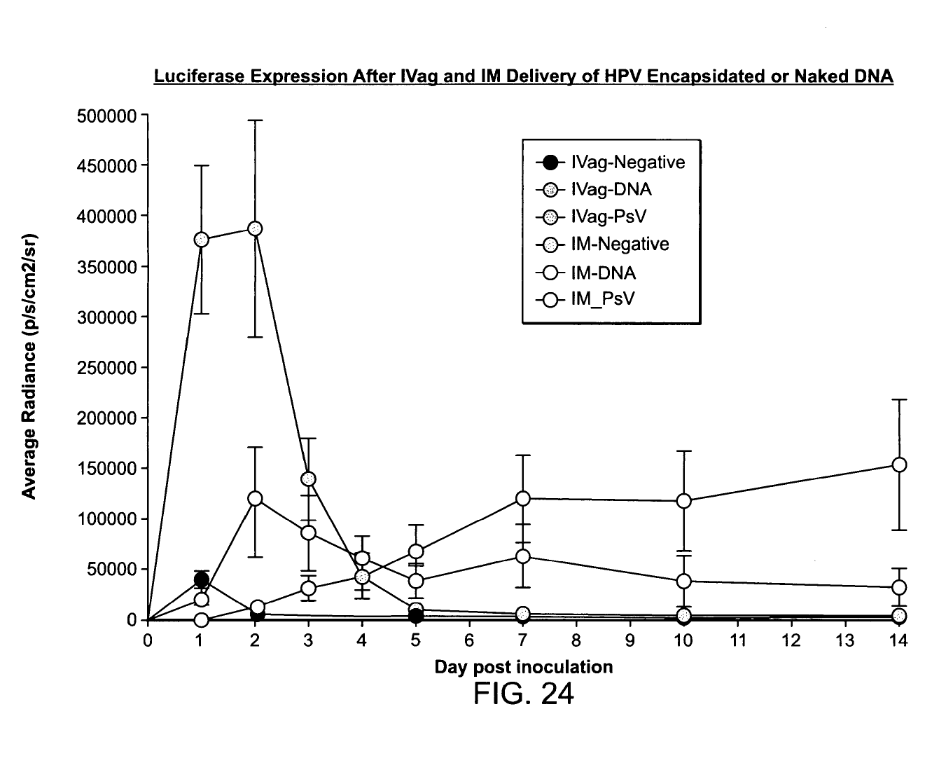

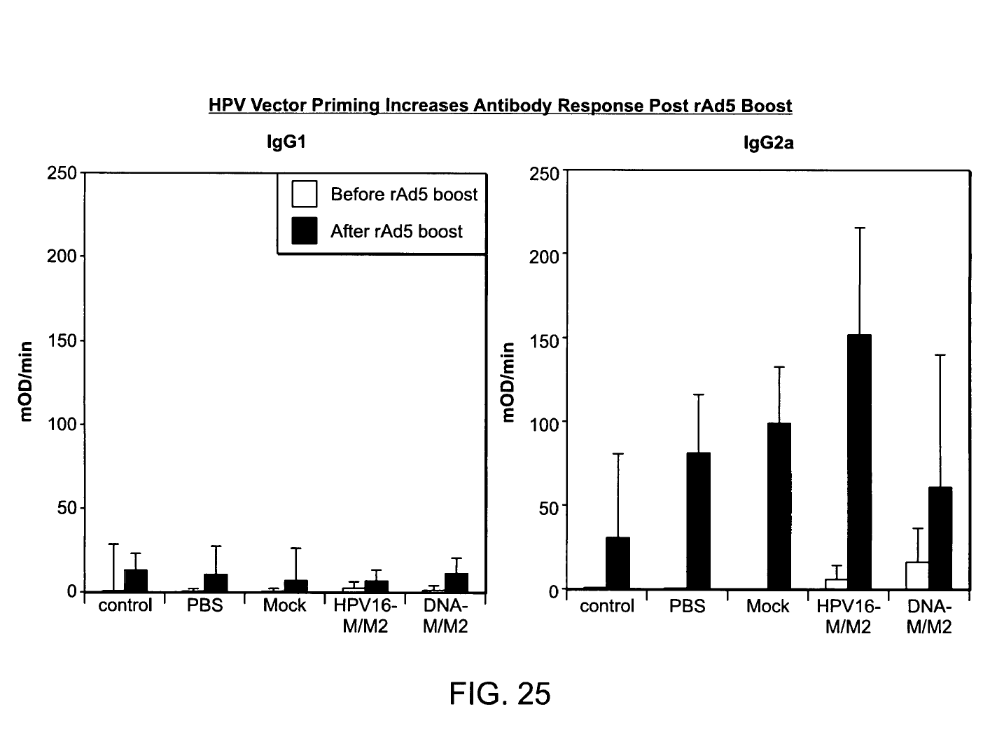

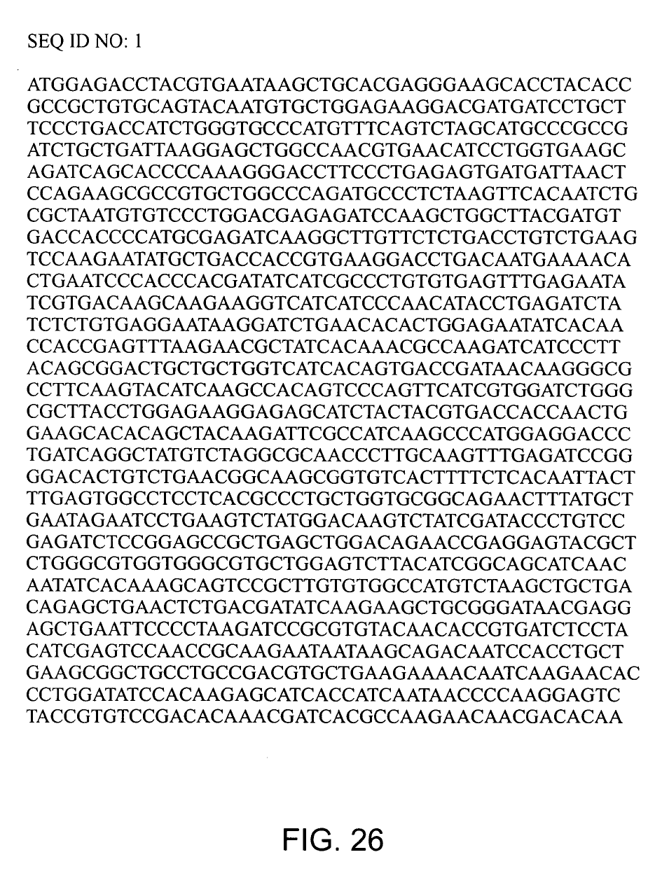

A fourth experiment was performed as outlined in FIG. 22. BALB/c mice were immunized with HPV16-M/M2, HPV45-M/M2, a range of naked DNA doses, or 1 μg of M/M2 protein with an empty plasmid. All mice received a single IVag inoculation after depoprovera and N-9 treatment. The experiment was performed to evaluate the potency of papillomaviral vector delivery of plasmid DNA relative to naked DNA. It is estimated that a dose of the HPV vector contains <5 ng of plasmid DNA. This experiment tested a 10,000-fold dose range of DNA from 5 ng to 50 μg. In addition, immunization with M/M2 purified protein assessed the possibility that the immune responses detected could be related to M/M2 protein contamination of the HPV vector preparations. The 1 μg dose is much higher than can be detected by Western blots in HPV vector preparations. Following RSV challenge, significant priming of the M2-specific CD8+ T cell response detected in lung by tetramer staining only occurred in the groups immunized with HPV16-M/M2 or HPV45-M/M2 (FIG. 23). These results indicate that multiple HPV serotypes can effectively package and deliver vaccine antigens expressed on DNA plasmids and that the HPV vector markedly enhances the potency of gene delivery.

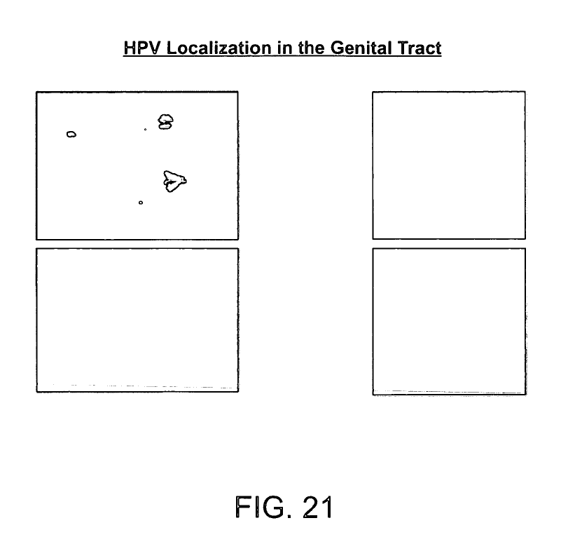

Because papillomaviral vectors can infect a broad range of different cell types in vitro, it was reasoned (and we have publicly suggested) that papillomaviral vectors might serve as effective genetic vaccine vehicles if administered by standard routes, such as intramuscularly. FIG. 21 shows HPV localization in the genital tract. However, we have found that intramuscular inoculation of mice with papillomaviral vectors carrying model antigens, such as M/M2, elicited poor immune responses compared to naked DNA-M/M2 and rAd5-M/M2 controls. In marked contrast, papillomaviral vectors delivered to disrupted vaginal epithelium induced unexpectedly robust immune responses to the delivered M/M2 gene, as well as against the papillomavirus capsid itself in inoculated mice. Mice inoculated intravaginally with papillomaviral vectors expressing M/M2 displayed systemic immune responses approaching those seen in mice administered adenoviral vectors expressing rAd5-M/M2) intramuscularly. This is remarkable in the sense that the naked DNA-M/M2 and Ad5-M/M2 systems have previously been optimized for generation of robust systemic immune responses in mice, whereas the papillomaviral vector immunogens have not been optimized.

These unexpected findings may be partly explained by a separate set of experiments, in which we have found that papillomaviral vectors are tropic for keratinocytes in vivo and appear to infect other tissues, such as skeletal muscle, less efficiently. Interestingly, intravaginal delivery of rAd5 and naked DNA vectors, which both exhibit broad tissue tropism, also induced unexpectedly robust immune responses if the vaginal epithelium was disrupted prior to inoculation.

In addition, prime-boost immunization regimens can be performed by the methods and examples as described herein. For example, vector priming may be carried out with HPV containing plasmids, in one case, and a boost regimen carried out with a different HPV serotype, or a different vector, such as rAd5, delivered mucosally. Thus, HPV-neutralizing antibody responses that might limit boosting could be avoided. It is also possible the HPV vector immunization could prime for other vaccine approaches such as protein or whole inactivated virus products.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method of eliciting an immune response in a subject comprising administering an immunogenic composition to an epithelial surface of the subject in combination with one or more agents or treatments to disrupt the epithelial surface, thereby eliciting an immune response in the subject.

2. The method of claim 1, wherein the method prevents or treats an infection or disease.

3. The method of claim 1, wherein the immunogenic composition comprises a viral vector, wherein the viral vector has encapsidated one or more nucleic acid segments, or fragments thereof, that encode proteins or fragments thereof.

4. The method of claim 3, wherein the viral vector is a papillomavirus vector or an adenovirus vector.

5. The method of claim 3, wherein the one or more nucleic acid segments, or fragments thereof, encode viral proteins or fragments thereof.

6. The method of claim 1, wherein the one or more agents or treatments to disrupt the epithelial surface is a chemical or mechanical agent or treatment.

7. The method of claim 5, wherein the nucleic acid segments, or fragments thereof, encode viral proteins selected from the group consisting of: Pneumovirus proteins, Papillomavirus proteins, Lentivirus proteins or Herpesvirus proteins.

8. The method of claim 7, wherein the Pneumovirus protein is Respiratory Syncytial Virus (RSV) protein is M, M2, N, F, SH, HN, E, or Gr.

9. The method of claim 7, wherein the Papillomavirus protein is Human Papilloma Virus (HPV) protein E1, E2, E4, E5, E6, or E7.

10. The method of claim 7, wherein the Lentivirus protein is Human Immunodeficiency Virus (HIV) protein, env, pol, gag, rev, nef, or tat.

11. The method of claim 7, where in the Herpesvirus protein is herpes simplex virus 1 or herpses simplex 2 protein gB, gC, gD, or gE.

12. The method of claim 7, wherein the one or more nucleic acid segments, or fragments thereof, comprise a fusion of the nucleic acid encoding a Pneumovirus codon-modified (M) matrix protein and codon-modified (M2) matrix protein.

13. The method of claim 7, wherein the one or more nucleic acid segments, or fragments thereof, comprise a fusion of a nucleic acid encoding a codon-modified (M) matrix protein and codon-modified (M2) matrix protein of RSV.

14. The method of claim 13, wherein the fusion of a nucleic acid encoding a codon-modified (M) matrix protein and codon-modified (M2) matrix protein comprises SEQ ID NO: 1.

15. (canceled)

16. An immunogenic composition for use in administration to a disrupted epithelial surface comprising a viral capsid, wherein the viral capsid comprises L1 and L2 proteins, and wherein the viral capsid has encapsidated one or more nucleic acid segments, or fragments thereof, that encode proteins or fragments thereof.

17. The immunogenic composition of claim 16, wherein the viral capsid is a papillomavirus capsid or an adenovirus capsid.

18-36. (canceled)

37. A nucleic acid molecule encoding any one of the genes, or fragments thereof, of claim 16.

38. An immunogenic composition comprising one or more of the vectors of claim 16, wherein the one or more nucleic acid segments, or fragments thereof, encode a viral surface protein and a papillomavirus capsid comprising L1 and L2 proteins, and wherein the immunogenic composition enhances protein expression and modulates an immune response.

39. An immunogenic composition comprising one or more of the vectors of claim 16, wherein the one or more nucleic acid segments, or fragments thereof, encode a viral surface protein and a adenoviral capsid comprising L1 and L2 proteins, and wherein the immunogenic composition enhances protein expression and modulates an immune response.

40. A kit for use in a method of eliciting an immune response in a subject, the kit comprising a viral capsid, wherein the viral capsid comprises L1 and L2 proteins, and wherein the capsid contains a vector comprising one or more nucleic acid segments, or fragments thereof, a pharmaceutically acceptable carrier, and instructions for use in administration to a disrupted epithelial surface.

41. The kit of claim 40, wherein the viral vector is a papillomavirus vector or an adenovirus vector.

42. (canceled)
